(19) United States
(12) Patent Application Publication
(10) Pub. No.: US 2013/0004581 A1
(43) Pub. Date: Jan. 3, 2013

(54) CONTROLLING UPTAKE BY CELLS

(75) Inventors: Rafi Korenstein, Tel-Aviv (IL); Nadav Ben-Dov, Moshav Ein-Ayala (IL)

(73) Assignee: Ramot at Tel-Aviv University Ltd., Tel-Aviv (IL)

(21) Appl. No.: 13/634,246

(22) PCT Filed: Mar. 17, 2011
(86) PCT No.: PCT/IB2011/051127

§ 371 Date: Sep. 12, 2012

Related U.S. Application Data
(60) Provisional application No. 61/282,708, filed on Mar. 19, 2010.

(51) Int. Cl. C12N 13/00; C12N 5/04; C12N 5/07; C12N 5/077; C12N 5/079; C12N 5/0735; C12N 5/0775; C12N 5/0797; C12N 5/09; C12N 15/85; C12N 15/82; C12N 15/86; C12M 1/42; A61K 33/00; A61K 35/14; A61N 1/04; C12N 5/078

(52) U.S. Cl. 424/529; 435/2; 435/410; 435/348; 435/375; 435/455; 435/468; 435/456; 435/173.4; 435/285.2; 435/283.1; 424/600; 607/115

(57) ABSTRACT

Methods and devices for causing uptake of materials by cells, temporary using local chemical environment modification. The modification may be caused chemically by reducing pH. The uptake method is passive and does not require bioactivity of the cells.

### RELATED APPLICATIONS

This application claims the benefit of priority under 35 USC 119(e) of a US provisional filing dated Mar. 19, 2010 and having Ser. No. 61/282,708, the disclosure of which is incorporated herein by reference.

### FIELD AND BACKGROUND OF THE INVENTION

The present invention, in some embodiments thereof, relates to controlling uptake of materials by cells and, more particularly, but not exclusively, to controlling uptake by pH modification.

Physical methods that induce uptake of molecules by cells include the application of a hypotonic stress (1), cell bombardment with coated particles (2), microinjection (3) and electroporation (4). Electroporation is associated with the application of high electric field. The electroporation process is defined by the formation of reversible high permeability state of the plasma membrane following the exposure of cells to high electric fields (in range of 400-600 V/cm). Due to the low conductivity of the cell lipid bilayer, application of external electric field generates a potential difference across the membrane and at a threshold value of about 200 mV, a sudden increase in membrane permeability is observed (5-7). These permeability changes are generally ascribed to the electric field induced formation of transient populations of hydrodynamic pores in the membrane, through which macromolecules can diffuse along their chemical or electrochemical gradients. Conditions required for efficient incorporation of macromolecules by electroporation are often associated with decrease in cell viability.

US patent applications and U.S. Pat. Nos. 5,964,726, 7,395,112, and Ser. Nos. 12/068,099, describe the application of electric fields to cells for therapy and/or causing material uptake.

Following are references possibly related to the description herein and/or referred to in the following text.
1. Poulin R, Coward J K, Lakanen J R and Pegg A E (1993) Enhancement of the spermidine uptake system and lethal effects of spermidine overaccumulation in ornithine decarboxylase-overproducing L1210 cells under hyposmotic stress. J Biol Chem 268, 4690-8.
2. Sanford J C, Smith F D and Russell J A (1993) Optimizing the biolistic process for different biological applications. Methods Enzymol 217, 483-509.
3. Soreq H and Seidman S (1992) Xenopus oocyte microinjection: from gene to protein. Methods Enzymol 207, 225-65.
4. Neumann E, Schaefer-Ridder M, Wang Y and Hofschneider P H (1982) Gene transfer into mouse lyoma cells by electroporation in high electric fields. Embo J 1, 841-5.
5. Ho S Y and Mittal G S (1996) Electroporation of cell membranes: a review. Crit. Rev Biotechnol 16, 349-62.
6. Valic B, et al. (2003) Effect of electric field induced transmembrane potential on spheroidal cells: theory and experiment. Eur Biophys J 32, 519-28.
7. Weaver J C (1995) Electroporation theory. Concepts and mechanisms. Methods Mol Biol 55, 3-28.
8. Antov Y, Barbul A and Korenstein R (2004) Electroendocytosis: stimulation of adsorptive and fluid-phase uptake by pulsed low electric fields. Exp Cell Res 297, 348-62.
9. Antov Y, Barbul A, Mantsur H and Korenstein R (2005) Electroendocytosis: exposure of cells to pulsed low electric fields enhances adsorption and uptake of macromolecules. Biophys J 88, 2206-23.
10. Motizuki M, et al. (2004) Low pH facilitates uptake of proteins by cells through a non-endocytic pathway. J Biochem 135, 713-9.
11. Harrison S C (2005) Mechanism of membrane fusion by viral envelope proteins. Adv Virus Res 64, 231-61.
12. Schibli D J and Weissenhorn W (2004) Class I and class II viral fusion protein structures reveal similar principles in membrane fusion. Mol Membr Biol 21, 361-71.
13. Bressanelli S, et al. (2004) Structure of a flavivirus envelope glycoprotein in its low-pH-induced membrane fusion conformation. Embo J 23, 728-38.
14. Bullough P A, Hughson F M, Skehel J J and Wiley D C (1994) Structure of influenza haemagglutinin at the pH of membrane fusion. Nature 371, 37-43.
15. Gibbons D L, et al. (2004) Conformational change and protein-protein interactions of the fusion protein of Semliki Forest virus. Nature 427, 320-5.
16. Kampmann T, et al. (2006) The Role of histidine residues in low-pH-mediated viral membrane fusion. Structure 14, 1481-7.
17. Modis Y, Ogata S, Clements D and Harrison S C (2004) Structure of the dengue virus envelope protein after membrane fusion. Nature 427, 313-9.
18. Sandvig K, Olsnes S, Petersen O W and van Deurs B (1987) Acidification of the cytosol inhibits endocytosis from coated pits. J Cell Biol 105, 679-89.
19. Boukamp P, et al. (1988) Normal keratinization in a spontaneously immortalized aneuploid human keratinocyte cell line. J Cell Biol 106, 761-71.
20. Anderle P, et al. (1998) P-Glycoprotein (P-gp) mediated efflux in Caco-2 cell monolayers: the influence of culturing conditions and drug exposure on P-gp expression levels. J Pharm Sci 87, 757-62.
21. Pontier C, et al. (2001) HT29-MTX and Caco-2/TC7 monolayers as predictive models for human intestinal absorption: role of the mucus layer. J Pharm Sci 90, 1608-19.
22. Iacopetta B J and Morgan E H (1983) The kinetics of transferrin endocytosis and iron uptake from transferrin in rabbit reticulocytes. J Biol Chem 258, 9108-15.
23. Weigel P H and Oka J A (1981) Temperature dependence of endocytosis mediated by the asialoglycoprotein receptor in isolated rat hepatocytes. Evidence for two potentially rate-limiting steps. J Biol Chem 256, 2615-7.
24. Huotari V, Vaaraniemi J, Lehto V P and Eskelinen S (1996) Regulation of the disassembly/assembly of the membrane skeleton in Madin-Darby canine kidney cells. J Cell Physiol 167, 121-30.
25. Yin H L and Stossel T P (1979) Control of cytoplasmic actin gel-sol transformation by gelsolin, a calcium-dependent regulatory protein. Nature 281, 583-6.
26. Yin H L, Hartwig J H, Maruyama K and Stossel T P (1981) Ca2+ control of actin filament length. Effects of macrophage gelsolin on actin polymerization. J Biol Chem 256, 9693-7.
27. Yin H L and Stossel T P (1980) Purification and structural properties of gelsolin, a Ca2+-activated regulatory protein of macrophages. J Biol Chem 255, 9490-3.
28. Yin H L, Zaner K S and Stossel T P (1980) Ca2+ control of actin gelation. Interaction of gelsolin with actin filaments and regulation of actin gelation. J Biol Chem 255, 9494-500.
29. Lamb J A, Allen P G, Tuan B Y and Janmey P A (1993) Modulation of gelsolin function. Activation at low pH overrides Ca2+ requirement. J Biol Chem 268, 8999-9004.
30. Chifflet S, et al. (2004) Effect of membrane potential depolarization on the organization of the actin cytoskeleton of eye epithelia. The role of adherens junctions. Exp Eye Res 79, 769-77.
31. Chifflet S, Hernandez J A, Grasso S and Cirillo A (2003) Nonspecific depolarization of the plasma membrane potential induces cytoskeletal modifications of bovine corneal endothelial cells in culture. Exp Cell Res 282, 1-13.
32. Nin V, Hernandez J A and Chifflet S (2009) Hyperpolarization of the plasma membrane potential provokes reorganization of the actin cytoskeleton and increases the stability of adherens junctions in bovine corneal endothelial cells in culture. Cell Motil Cytoskeleton 66, 1087-99.
33. Flewelling R F and Hubbell W L (1986) The membrane dipole potential in a total membrane potential model. Applications to hydrophobic ion interactions with membranes. Biophys J 49, 541-52.
34. Dejonge P C, et al. (1983) Phloretin—an Uncoupler and an Inhibitor of Mitochondrial Oxidative-Phosphorylation. Biochimica Et Biophysica Acta 722, 219-225.
35. Reyes J and Benos D J (1984) Changes in Interfacial Potentials Induced by Carbonylcyanide Phenylhydrazone Uncouplers—Possible Role in Inhibition of Mitochondrial Oxygen-Consumption and Other Transport Processes. Membrane Biochemistry 5, 243-268.
36. Mosiman V L, Patterson B K, Canterero L and Goolsby C L (1997) Reducing cellular autofluorescence in flow cytometry: an in situ method. Cytometry 30, 151-6.
37. Halliwell B and Whiteman M (2004) Measuring reactive species and oxidative damage in vivo and in cell culture: how should you do it and what do the results mean? Br J Pharmacol 142, 231-55.
38. Wilson J X (2005) Regulation of vitamin C transport. Annu Rev Nutr 25, 105-25.

### SUMMARY OF THE INVENTION

A broad aspect of some embodiments of the invention relates to controlling uptake by controlling local chemical environment, for example, by controlling hydrogen ion availability, which hydrogen ion availability may be made, for example, by electrical or chemical means and/or which hydrogen ion availability may directly cause the invaginations and/or vesiculation in the plasma membrane of living cells.

Described below are experimental findings showing that elevation of hydrogen ion concentration in the extracellular compartment induces the uptake of soluble molecules and/or particles. The findings show that proton induced uptake (PIU) (also termed herein LpHU—low pH uptake) is independent of ATP availability and can occur at an almost a constant rate. Moreover PIU, in some cases, is only partially sensitive to temperature as low as 4° C. It is also shown that uptake events are sensitive to cell membrane polarization (Δψ) but not to trans-membrane hydrogen gradient (ΔpH).

Evidence is presented that the PIU is accompanied by the formation of invaginations and/or vesicles, at least in the initial phase, followed by release of entrapped molecules into the cytosol; dextran probes are found in a pH environment similar to the cytosolic one and gold-labeled IgG are seen aggregated within the cells in images taken by electron microscopy.

It is hypothesized, that at least in some embodiments, the excess hydrogen ions cause an increase in curvature of the cell membranes, which causes invagination. External environment which is near invagination can then enter the invagination. Possibly, the rigidity of the cell membrane, caused, for example, by membrane proteins and/or carbohydrates, causes the invagination to close on itself, creating an intra-cellular vesicule with encapsulated external environment. When this vesicle is compromised, the external environment is delivered to the interior of the cell.

There is provided in accordance with an exemplary embodiment of the invention a method of inducing uptake in living cells, comprising:
determining a desired uptake of a material by the cells; and
in response to said determining, temporarily subjecting said cells to a local chemical environment which encourages inward vesiculation or invagination of a plasma membrane of the living cells causing said uptake and which does not substantially affect said cells by osmotic effects, said subjecting being for a period of less than 6 hours.

In an exemplary embodiment of the invention, said encouraging local chemical environment comprises a reduction in pH which is not local to the membrane of the cells. Optionally or alternatively, said encouraged inward vesiculation comprises vesiculation caused by a chemical effect and not by a biochemical effect involving the chemical activity of proteins. Optionally or alternatively, subjecting comprises intentionally subjecting to an environment which causes substantial cell death when subjecting for a period greater than said period. Optionally or alternatively, the method comprises selecting method parameters in accordance with a pH mediated uptake mechanism. Optionally or alternatively, said subjecting comprises controlling an uptake rate of said cells by controlling said local chemical environment. Optionally or alternatively, said subjecting comprises avoiding damaging the living cells by said uptake and by materials being taken up. Optionally or alternatively, said subjecting comprises avoiding damaging the living cells by said uptake.

In an exemplary embodiment of the invention, said subjecting comprises applying an anodic current for a time and amount sufficient to cause acidification by anodic hydrolysis of water, to said cells to provide or modify said encouraging chemical environment, said current not being sufficient to cause substantial electroporation. Optionally,

applying comprises applying a voltage and current density sufficient for electrolysis for a duration of between 1 second and 15 minutes. Optionally or alternatively, applying comprises controlling an applied pH to avoid applying a pH to cells below a desired level, by positioning of an anode.

In an exemplary embodiment of the invention, said local chemical environment comprises an increase in hydrogen ions. Optionally, said environment has a pH value between 3 and 6. Optionally or alternatively, said increase is to above physiological concentrations of hydrogen ions, for a time period which does not kill more than 25% of said cells. Optionally, said increase is to above physiological concentrations of hydrogen ions, for a time period which does not kill more than 10% of said cells.

In an exemplary embodiment of the invention, said increase is provided by one or more of:
a. adding a soluble formulation of acidic material;
b. release of hydrogen ions from solid, semi-solid or liquid substances;
c. release of hydrogen ions from a proton exchange membrane (PEM); and
d. chemical cleavage of molecules to release hydrogen ions.

In an exemplary embodiment of the invention, said living cells are inside a living body. Optionally, said local environment is effected by controlling a physiology of said body.

In an exemplary embodiment of the invention, said local environment is effected by the provision of one or more formulations to said environment. Optionally, said formulations include a hydrogen ion releasing formulation. Optionally or alternatively, said provision is substantially limited to said environment.

In an exemplary embodiment of the invention, the method comprises providing at least one agent to be introduced into said cells by said uptake. Optionally, said agent comprises a simple molecule formulation. Optionally or alternatively, said agent comprises a nanoparticle. Optionally or alternatively, said agent is selected from a group consisting of:
a. a nucleic acid agent;
b. a small molecule agent;
c. a protein aceous agent; and
d. a carbohydrate agent;
e. lipid agent; and
f. a combination of same.

Optionally, said nucleic acid agent comprises a nucleic acid expression constructs.

Optionally, said nucleic acid agent nucleic comprises an expression silencing agent. Optionally, said expression silencing agent is selected from the group consisting of an siRNA, a miRNA, an antisense, a ribozyme and a DNAzyme.

In an exemplary embodiment of the invention, said agent is selected from the group consisting of an enzyme, an antibody, a toxin, a hormone, a growth factor, a ligand, a structural protein and a fluorescent protein.

Optionally, said small molecule agent comprises a drug.

Optionally, said agent comprises an identifiable moiety.

Optionally, said agent comprises a therapeutic moiety.

In an exemplary embodiment of the invention, the method comprises inducing said uptake for one or more of the following purposes:
a. interacting with cell functions including one or more of enzymes, catalytic domains and respiration chain components;
b. incorporate toxins, peptides, proteins, fatty acids, inhibitors, blockers or promoters;
c. introducing to said cells new properties, new functions, correcting resident mutations or silencing existing functions;
d. interfering with protein expression and cell functioning, for example anti-sense RNA and siRNA;
f. labeling structures in the cell;
g. identifying biological pathways; and
h. inducing cell proliferation, growth arrest or cell killing.

Optionally, the method comprises selecting said formulation to have a desired therapeutic effect.

In an exemplary embodiment of the invention, said formulation includes an agent for targeting a specific intracellular part of said living cells.

In an exemplary embodiment of the invention, the method comprises maintaining a desired level of said formulation in said living cells, by said uptake.

In an exemplary embodiment of the invention, the method comprises maintaining a desired level of said formulation in said living cells, by controlling said local environment.

In an exemplary embodiment of the invention, the method comprises maintaining a desired level of said formulation in said living cells, by interfering with expulsion of said formulation from said cells.

In an exemplary embodiment of the invention, said cells are red blood cells.

In an exemplary embodiment of the invention, said cells are white blood cells.

In an exemplary embodiment of the invention, the method comprises repeating said temporarily subjecting a plurality of times to achieve a total desired uptake.

In an exemplary embodiment of the invention, said duration is less than 2 hours. Optionally, said duration is less than 30 minutes. Optionally, said duration is less than 5 minutes.

In an exemplary embodiment of the invention, the method comprises modifying a mechanical stiffness of said cells for said uptake.

In an exemplary embodiment of the invention, the method comprises modifying a membrane polarization of said cells for said uptake.

In an exemplary embodiment of the invention, determining comprises calculating and imposing a chemical environment designed to provide said uptake to within a factor of 4 of said desired uptake.

In an exemplary embodiment of the invention, said encouraging local chemical environment comprises a reduction in pH which is local to the membrane of the cells.

In an exemplary embodiment of the invention, the method comprises modifying a buffering capacity or a pH of a local fluid in addition to applying said current.

In an exemplary embodiment of the invention, said environment has a pH value between 4 and 5.5.

In an exemplary embodiment of the invention, said uptake is at least 5 times an uptake in a neural chemical environment. Optionally, said uptake is at least 10 times an uptake in a neural chemical environment. Optionally, said uptake is at least 20 times an uptake in a neural chemical environment. Optionally, said uptake between 30 and 100 times an uptake in a neural chemical environment.

There is provided in accordance with an exemplary embodiment of the invention method of treating a body portion, comprising providing a formulation to said body, selected to be differentially and meaningfully taken up by said portion relative to another portion, based on an expected pH of said portion relative to a pH of the another portion.

Optionally, said portion is selected from a group comprising a tumor, an ulcer, lymphocytes, erythrocytes, blood vessels, bones, spleen, pancreas, pulmonary, and muscles.

There is provided in accordance with an exemplary embodiment of the invention, apparatus for controlling cellular uptake, comprising:
at least one electrode;
a power source, electrifying said at least one electrode as an anode; and
a controller adapted to control said power source according to a pH-mediated uptake protocol. Optionally, the apparatus comprises a pH sensor. Optionally or alternatively, the apparatus comprises a tissue displacer located adjacent said electrode.

In an exemplary embodiment of the invention, said controller is configured to estimate an uptake effect based on said protocol.

In an exemplary embodiment of the invention, the apparatus comprises a source of formulation for said uptake.

There is provided in accordance with an exemplary embodiment of the invention, a method of processing blood, comprising:
providing blood;
causing uptake of material into cells of said blood using a pH mediated effect;
stopping said uptake.

Optionally the method comprises returning said treated blood to a body from which it was taken.

There is provided in accordance with an exemplary embodiment of the invention, human blood cells loaded with a material not found in said blood cells and incapable of self-transport through a membrane and having a molecular weight of at least 70 kD.

There is provided in accordance with an exemplary embodiment of the invention, a tissue culture including an acidifying material in an amount sufficient to cause an uptake of a second material without substantially damaging said tissue.

There is provided in accordance with an exemplary embodiment of the invention, the use of an acidifier to control uptake into living cells, for example, substantially as described herein.

There is provided in accordance with an exemplary embodiment of the invention, the use of electrically generated protons to control uptake into living cells, for example, substantially as described herein.

There is provided in accordance with an exemplary embodiment of the invention, a method of electrical modification of cells, comprising determining a field to apply in order to have a desired effect, based on a chemical environment of said cells.

There is provided in accordance with an exemplary embodiment of the invention, a method of loading human blood cells with a material, comprising first causing a first uptake of material by said cells and thereafter causing a second uptake of material, without separating out damaged blood cells.

There is provided in accordance with an exemplary embodiment of the invention, a method of loading human blood cells with a material, comprising exposing said cells to a material; and causing said material to be taken up while damaging fewer than 10% of said cells by said uptake, while causing uptake in at least 50% of said cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C are chats showing the dependence of PIU on extracellular pH, in accordance with some embodiments of the invention. Adherent cell cultures (HaCaT, Caco-2/TC7, COS 5-7 and HT29/mtx) and suspended cultures (TK6) were exposed to solutions of different pH in the presence of dextran-FITC for a period of 5 minutes, before being washed, harvested and analyzed. Uptake, based on flow cytometry, is plotted as function of the external pH from 3 independent experiments per cell line. In

FIG. 1A is shown the Extent of uptake in terms of FITC intensity geometrical mean±SD is presented as fold induction relative to the constitutive uptake at physiological pH 7.4. In FIG. 1B is shown the PI (a necrotic cell marker) stained cells fraction±SD (%) relative to unexposed control population. In FIG. 1C is shown the FITC geometrical mean±SD presented as fold induction relative to the constitutive uptake at physiological pH 7.4. At pH 6, difference in dextran concentration among the cell lines is nearly significant (P=0.056 by one way ANOVA). At pH 5, HaCaT and TK6 have 2 folds higher dextran concentration then Caco2/TC7 and COS 5-7 (P<0.001 t-test), which in turn have 2 folds higher dextran concentration than HT29 cells (P<0.001 t-test). This illustrates the basic mechanism of uptake and can be used, in some embodiments of the invention, to assess effect of pH on uptake.

FIGS. 2A-2B are charts illustrating kinetics of PIU, in accordance with some embodiments of the invention. For FIG. 2A, Caco2/TC7 cells were harvested and suspended in HBSS. The cells were exposed to solutions of varying pH (7.4, 6.4, 6, 5.5 and 5) in the presence of dextran-FITC for 6 different time periods (1, 5, 10, 20, 30 and 60 minutes). FACS analyses (FL1) of uptake in terms of geometrical mean±SD are presented as fold induction relative to the constitutive uptake at physiological pH 7.4, from 5 independent experiments (n=15). For FIG. 2B the efflux following PIU was measured in Caco2/TC7 cultures that were harvested and suspended in HBSS before being exposed to pH 5.3 solutions for duration of 10 minutes in the presence of dextran-FITC. The cultures were washed in fresh HBSS and divided into groups incubated at either 37° C. or 4° C. for four time periods (15, 60, 120 and 180 minutes). Results from FACS analysis (FL1) geometrical mean±SD are presented as fold induction relative to the fluorescent intensity of those cells analyzed 15 minutes after solution pH was neutralized. n=9 in 3 independent experiments. These kinetics can be used, in some embodiments of the invention, to determine desired uptake.

FIG. 3 is a chart of the uptake of dextran-FITC during a 10 minutes exposure to external pH. Cells were exposed to low pH solution for 10 minutes followed by buffering and washing. Uptake is measured by FACS and results are given as folds of geometrical mean±SD relative to the constitutive uptake. This can be used, in some embodiments of the invention, to assess effect of pH on uptake.

FIGS. 4A-4B are charts showing the rate of PIU in cells pretreated with MDR inhibitors or ATP depletion, in accordance with exemplary embodiments of the invention. For FIG. 4A HaCaT cultures were harvested and suspended in HBSS before treated by 50 μM Cyclosporin-A, 20 μM verapamil or with 0.1% DMSO for the control group. The cultures were exposed to a pH 5.25 solution along with dextran-FITC for 3 time periods (5, 15 and 30 minutes). FACS analyses (FL1) in terms of geometrical mean±SD are presented as fold induction relative to the constitutive uptake at pH 7.4. The linear regression among the exposure times in the control cultures is poor (R²=0.87), comparing to the linear regression of cultures treated with cyclosporin-A or verapamil (R²=0.99 for both). For FIG. 4B ATP depleted and non-depleted HaCaT cells were exposed to either pH 5.25 or pH 7.4 for up to 30 minutes duration in the presence of dextran-FITC at 24° C. Cells were harvested and suspended in PBS in the presence of with 0.1% azide, 6 mM iodocetamide and 10 mM inosin for one hour in 37° C., leading to 97% of decrease of intracellular ATP, as verified by the luciferin-luciferase assay. PIU determined by FACS analysis (FL1) in terms of geometrical mean±SD is presented relative to constitutive uptake at pH 7.4. In the charts, * indicates a t-test p<0.05; and ** indicates a t-test p<0.001. This illustrates the basic mechanism of uptake and can be used, in some embodiments of the invention, to assess effect of blocking of cell metabolism and/or expelling on uptake.

FIGS. 5A-5C are charts showing the dependence of PIU on temperature, in accordance with exemplary embodiments of the invention. For FIG. 5A HaCaT cells were harvested, suspended in HBSS and incubated at either 24° C. or 4° C. Next, the cells were exposed for up to 60 minutes to pH 5.25 or pH 7.4 both in the presence of dextran-FITC at these solution temperatures throughout the length of the experiment. FACS analyses (FL1) in terms of geometrical mean±SD are presented as fold of induction relative to constitutive uptake (pH 7.4) under each temperature. n=12 in 4 independent experiments. Fir FIG. 5B HaCaT cells were harvested and suspended in HBSS solution at the following temperatures: 4° C., 9° C., 12° C., 16° C., 20° C., 24° C., 28° C. or 36° C. The cells were incubated at the designated temperature for 15 minutes before they were exposed to pH 5.25 or pH 7.4 for 10 minutes in the presence of dextran-FITC. The linear regression is R²=0.99. FACS analyses in terms of geometrical mean±SD are presented as fold induction relative to the constitutive uptake at pH 7.4. n=15 in 3 independent experiments. FIG. 5C shows the plotting of the Arrhenius relationship of the PIU rate natural logarithm against the inverse absolute temperature. This can be used, in some embodiments of the invention, to assess effect of temperature on uptake.

FIG. 6 is a chart showing PIU of molecules different by size and charge, in accordance with some embodiments of the invention. Hacat cultures were exposed to solutions of pH 5.2 or pH 7.4 for 5 minutes period, in the presence of different fluorescence molecules (All at 25 μM concentration). FACS analysis results (FL1, geometrical mean±SD) for the cell cultures exposed to pH 5.2 are presented as fold induction relative to results obtained for cells exposed to pH 7.4. This can be used, in some embodiments of the invention, to assess effect of molecule size and charge on uptake.

FIGS. 7A-7B are charts showing the comparison of PIU in adherent and suspended HaCaT cells, in accordance with some embodiments of the invention. For FIG. 7A, HaCaT cells were tested either at their adherent state, or following harvest at a suspended state. The cells from both groups were exposed in parallel to pH 5.25 for five durations (5, 15, 25, 35 and 65 minutes). Dextran-FITC was added for the last 5 minutes of every exposure. FACS analyses (FL1) in terms of geometrical mean±SD are presented as fold induction relative to the constitutive uptake at physiological pH 7.4. n=9 in 3 independent experiments. For FIG. 7B, HaCaT Cells were cultivated at low, medium or high cell density. The adherent cultures were exposed for 10 minutes to solutions of varying pH (7.4, 6, 5.4 and 4.6) in the presence of dextran-FITC. The cultures were harvested and undergone FACS analyses (FL1) in terms of geometrical mean±SD and are presented as fold induction relative to the constitutive uptake of low density cultures. Statistical significance was tested against the confluent culture, n=9 in 3 independent experiments. In the charts, * indicates a t-test p<0.05; and ** indicates a t-test p<0.01. This can be used, in some embodiments of the invention, to assess effect of cell membrane rigidity and/or tissue structure on uptake.

FIG. 8 is a chart showing a correlation between dextran PIU and external dextran concentration, in accordance with some embodiments of the invention. HaCaT cells cultures were exposed to pH 5.2 or pH 7.4 in the presence of dextran-FITC (70 kDa) for a period of 5 minutes. FACS analysis results for the cell cultures fluorescence are presented as geometrical mean±SD against the external concentration of dextran. This can be used, in some embodiments of the invention, to assess effect of molecule concentration on uptake.

FIGS. 9A-9E are images and charts which illustrate cell associated dextran-FITC fluorescence in response to extracellular pH, in accordance with some embodiments of the invention. COS 5-7 cells, cultivated in glass bottom 96 wellplate, were exposed to a pH 5.25 solution containing 70 kD dextran-FITC (5 μM) for 15 minutes period, followed by washing the culture with K⁺PBS solution (130 mM potassium) at pH7.4. FIG. 9A shows cultures incubated in K⁺PBS at pH 7.4. FIG. 9B shows cultures incubated in K⁺PBS at pH 6. FIG. 9C shows cultures incubated in K⁺PBS at pH 6 with 10 μM Nigiricin. Microscopic images were acquired in fluorescence (ex485/em530) and DIC channels at ×100 magnification. For FIG. 9D HaCaT cells were harvested and suspended in HBSS and exposed to pH 7.4 or 5.25 in the presence of dextran-FITC for 10 minutes, washed and re-suspended in high potassium buffer (130 mM K⁺) at pH 7.4. Cells suspension was divided into three subgroups, each subgroup analyzed by FACS. The medium in each of the subgroups was titrated to pH 7.1, pH 6.6 or pH 5.9, followed by second FACS analysis. Next, each of the subgroups received 10 μM Nigericin and analyzed again by FACS. Results are given as folds of geometrical mean±SD relative to the FITC intensity of the cells in pH 7.4 from 2 independent experiments (n=12). * indicates a t-test p<0.05; and ** indicates a t-test p<0.01. For FIG. 9E Intracellular pH response following decreased of extracellular pH level. Caco2 cells loaded with BCEFC are suspended in HBSS solutions. Solution pH was altered by 10 mM MES and titrated with HCl. The cytosolic pH level was determined from BCECF fluorescent intensity using the ratiometric method in 12 independent measurements. This can be used, in some embodiments of the invention, to assess effect of pH on uptake.

FIGS. 10A-10B are images which illustrate the adsorption and uptake of polystyrene nanoparticles and phalloidin, in accordance with some embodiments of the invention. Cells in suspension were incubated with 60 nm polystyrene particles (Red and Blue) for 15 minutes either at pH 7.4 or pH 5, washed and stained with green fluorescent membrane stain. Images of live cell optical cross sections were acquired by Leica SCLM in the blue (420 nm), green (520 nm) and the red (600 nm) emission channels. FIG. 10A shows a composite channels image of a cell exposed to pH 5. FIGS. 10A1, 10A2 and 10A3 depict the green, red and blue channels respectively. FIG. 10B shows a composite channels image of a cell exposed to pH 7.4 and treated by the same procedure. This can be used, in some embodiments of the invention, to assess effect of pH on uptake.

FIGS. 11A, 11B, 11C, 11D, 11E, 11F, 11D1, 11E1, 11F1, 11G, 11H, 11K, 11L, 11L1, 11L2, 11M1, 11M2, 11M3 are transmission electron microscope images of cells during or following exposure to low pH. HaCaT cells were harvested and suspended in HBSS. The cells were exposed to pH 5 for 10 minutes and later fixated with Karnovsky solution. Post process includes OsO₄ fixation, dehydration and embedding in glycid ether. Thin sections stained with uranyl-acetate and lead-citrate, were examined in Jeol 1200EX transmission electron microscope. FIGS. 11A-11B show unexposed cells maintained at pH 7.4. FIGS. 11C-11K show cells fixated while exposed to pH 5. FIG. 11L shows cells fixated 10 minutes after the end of acidic exposure. Images show clusters of small round structures that appear empty, near the perimeter of the cells cross section (arrows in FIG. 11C), which cannot be seen in cells not exposed to acidic environment (FIGS. 11A and 11B). In virtually all of the cells exposed to a low pHₑₓₜ 5, such clusters could be detected at discrete areas (FIGS. 11D, 11E and 11F) at the cell perimeter. Detailed images of such areas (FIGS. 11D1, 11E1 and 11F1 at X50,000) reveal that these vesicles are either in direct contact with the plasma membrane or at its immediate proximity. At areas away from the plasma membrane, some of the vesicles begin to fuse together (white arrows). The apparent uniformity of the vesicular structures suggests the shape of round small vesicles rather then cross sections of elongated tubule. Higher resolution images taken in the proximity of the plasma membrane (FIGS. 11G, 11H and 11K at X100,000) show these vesicular structures to either “kiss” the inner side of the membrane (small white arrows) or to form buds-like protrusions (large bold arrows). Images of cells fixated 10 minutes after the cells were brought back to pHₑₓₜ 7.4, reveal different scenery. There are fewer cells seen with vesicular clusters (FIG. 11L), and from those that can be seen, large proportions appear to be already fused together, accompanied by vesicles still undergoing fusion (large arrows in FIG. 11L1 at X50,000). Higher resolution also reveals the presence of vesicles touching at the inner side of the plasma membrane (small arrows, FIG. 11L2 at X100,000). In FIG. 11M2 the arrow points to gold nanoparticles entrapped inside folds of plasma membrane following PIU. FIGS. 11M1 and 11M3 portray aggregated gold nanoparticles that escaped into the cytoplasm. This can be used, in some embodiments of the invention, to assess effect of pH on uptake.

FIGS. 12A-12B are a chart and images which show the relative change of PIU by modifiers of actin cytoskeleton organization, in accordance with some embodiments of the invention. For. FIG. 12A HaCaT cells were treated with the following cytoskeleton modifiers: latrunculin-A (1 μM), cytochalasin-B (10 μM), wortmannin (1 μM), calyculin-A (50 nM) or exposed to pH 5.25 for 15 minutes before they were exposed to pH 5.25 or 7.4 for additional 10 minutes, along with 70 kD dextran-FITC (5 μM). FACS analyses in terms of geometrical mean±SD are presented as folds of induction relative to the constitutive uptake found for every group. n=12 in 4 independent experiments. * indicates a t-test p<0.05; and ** indicates a t-test p<0.01FIGS. 12Ba-12Bf show Fluorescent images of adherent HaCaT cultured on glass cover-slips: FIG. 12Ba without additional treatment; FIG. 12Bb exposed to low pH; FIG. 12Bc treated by cytochalasin-B; FIG. 12Bd treated by wortmannin; FIG. 12Be treated by latrunculin-A; and FIG. 12Bf treated by calyculin-A. Following treatment, the cells were fixed with 4% paraformaldehyde and stained with phalloidin-TRITC. Images were acquired using fluorescent microscope employing an Ex540 nm/Em580 nm filter at ×100 magnification. This can be used, in some embodiments of the invention, to assess effect of cell membrane rigidity and/or co-applied materials on uptake.

FIGS. 13A-13Bc area chart and images which show the dependence of the PIU and actin organization on modification of the transmembrane potential difference, in accordance with some embodiments of the invention. For FIG. 13A the cultures' medium was replaced with HBSS modified by replacing sodium ions with other ions, while preserving the solution iso-osmolarity. The cultures were exposed to pH 5.25 or 7.4 for duration of 10 minutes, before dextran-FITC (43 kD, 5 μM) was added for additional 5 minutes. FACS analysis results (FL1, geometrical mean±SD) for the cell groups exposed to modified solutions are presented as fold induction relative to results obtained for cells exposed in unmodified HBSS. n=9 in 3 independent experiments. * indicates a t-test p<0.05; and ** indicates a t-test p<0.01. For FIGS. 13Ba-13Bc HaCaT cultures were cultured on glass cover-slips: Culture medium was replaced by PBS (FIG. 13Ba), potassium free PBS (FIG. 13Bb) or 130 mM potassium PBS (FIG. 13Bc). The cells were fixated by 4% paraformaldehyde and stained with phalloidin-TRITC, and images were acquired using fluorescent microscope employing Ex540 nm/Em580 nm filter. This can be used, in some embodiments of the invention, to assess effect of membrane polarization and/or desirability thereof on uptake.

FIG. 14 is a chart which shows the dependence of PIU on a molecular agent that reduce the plasma membrane dipole potential, in accordance with some embodiments of the invention. Phloretin (25 μM) or CCCP (25 μM) were added to HaCaT cell cultures 10 minutes prior to PIU treatment. The cells were exposed to pH 5.2 or 7.4 in the presence of dextran-FITC (70 kDa) for 10 minutes period. FACS analysis results (FL1, geometrical mean±SD) for the cell groups exposed to pH 5.2 are presented as fold induction relative to results obtained for cells exposed to pH 7.4. n=9 in 3 independent experiments. This can be used, in some embodiments of the invention, to assess effect of membrane dipole potential and/or desirability thereof on uptake.

FIG. 15 is an upper view of an exposure chamber used in some experiments described herein.

FIG. 16 is a chart showing uptake by cells exposed to LEF (low electric field) in a three compartment exposure device, as in FIG. 15, in accordance with an exemplary embodiment of the invention. LEF was applied to COS 5-7 suspensions, containing Dextran-FITC (100 μM) by employing Pt electrodes in the three compartment exposure device at 24° C. Uptake analyzed by FACS is given as folds of geometrical mean±SD relative to the constitutive uptake. n=9 in 3 independent experiments. * indicates a t-test p<0.05; and ** indicates a t-test p<<0.01. This can be used, in some embodiments of the invention, to assess effect of electric fields on uptake.

FIG. 17 is a chart of the extent of dextran-FITC uptake as a function of LEF of various field strength and current density, in accordance with some embodiments of the invention. Uptake was carried out at constant electric field strength (20 V/cm) and at four different current densities was statistically different (one-way ANOVA p<0.05). Uptake within each current density (200 mA/cm², 160 mA/cm², 120 mA/cm² and 80 mA/cm²) is fairly constant (one-way ANOVA p>0.05) independent of electric field strength. Medium conductivity was varied by replacing salts with sucrose while maintaining constant osmolarity. Uptake was measured by FACS (FL1) and results are given as folds of geometrical mean±SD relative to the constitutive uptake. n=12 in 4 independent experiments. This can be used, in some embodiments of the invention, to assess effect of electric fields parameters on uptake.

FIG. 18 is a chart of intracellular oxidative stress in the presence of anti-oxidants in the extracellular and the intracellular compartments, in accordance with some embodiments of the invention. The intracellular oxidative stress measured by intracellular DCF fluorescent intensity following cell exposure to electric pulse train, analyzed by FACS (488/530 nm). Results are given as folds of geometrical mean±SD relative to the level of constitutive oxidative stress. Statistical t-test results: 2 mM SAA; p<0.05, 1 mM DHA; p<0.01. This can be used, in some embodiments of the invention, to assess effect of and/or desirability of anti-oxidants and/or electric fields on uptake.

FIG. 19 is a chart of the dependence of electric induced uptake of dextran-FITC on the presence of anti-oxidants in the extracellular and the intracellular compartments, in accordance with some embodiments of the invention. Uptake is measured by FACS (488/530 nm) and results are given as folds of geometrical mean±SD relative to the constitutive uptake. No statistical difference is found between all groups using one-way ANOVA (p>0.05).

FIGS. 20A-20B are images showing the spatial profile of hydrolytic induced low pH near the anode interface at different buffer capacities, in accordance with some embodiments of the invention. A series of pH sensitive paper indicators are portrayed side by side, where each paper indicates the level of transient pH in the anode compartment during exposure to electric fields, such as an ECT (electric current train), on HBSS media supplemented with HEPES in the range of 60-100 mM. The paper is dipped perpendicular to the anode surface and pulled out in one quick movement, thus paper exposure to the solution is longer at its bottom section. In FIG. 20A the green color beginning from the lower left corner is the paper's indicator response to pH value of 1.0-2.0 while the blue color indicates response to pH>5.0. In FIG. 20B a different pH indicator is used where the yellow color indicates response to pH<5.0 while the violet color indicates pH≦7.0. This can be used, in some embodiments of the invention, to assess effect of electric fields on uptake.

FIG. 21 is a chart of the dependence of EPT-induced uptake of dextran-FITC on medium's buffer capacity, in accordance with some embodiments of the invention. Cells were exposed to ECT in HBSS with varying concentration of HEPES and upon termination of ECT were immediate diluted in a DMEM-H buffer. Uptake was measured by FACS (FL1) and results are given as folds of geometrical mean±SD relative to the constitutive uptake. n=18 in 6 independent experiments.

FIG. 22 is a chart illustrating the uptake of siRNA molecules by cells following repeated exposure to low pH solution, in accordance with some embodiments of the invention. HeLa cells were incubated with siRNA (HIF1α) in low pH solution or in control solution (PBS). The cells were exposed to siRNA solution for periods of one hour interrupted by two hours periods of incubation in growth medium. The number of siRNA molecules in the cells was quantified using RT-PCR. This can be used, in some embodiments of the invention, to set parameters for siRNA therapy.

FIGS. 23A1-23B3 are images which illustrate cell uptake of dextran-FITC following exposure to low pH, in accordance with some embodiments of the invention. In FIGS. 23A1-A3 COS 5-7 cultures were cultivated on glass cover slip and incubated with dextran-FITC for a 15 minutes period at two different pH values. Microscopic images were acquired with Zeiss fluorescent microscope by fluorescence (ex485/em530) and DIC channels: FIG. 23A1 shows culture maintained at pH 7.4; FIG. 23A2 shows culture exposed to pH 5.25 and visualized immediately after exposure; and FIG. 23A3 shows culture exposed to pH 5.25, washed and incubated for additional 15 minutes in cold DMEM-H in the absence of dextran-FITC. In FIGS. 23B1-B3 COS 5-7 cells were harvested and suspended in HBSS. Cells suspensions were incubated with 43 kD dextran-FITC (10 μM) for 15 minutes. Microscopic images were acquired using Leica SCLM at the FITC and DIC channels: FIG. 23B1 shows a cell maintained at pH 7.4; and FIGS. 23B2 and 23B3 show cells exposed to pH 5.25. This can be used, in some embodiments of the invention, to assess effect of pH on uptake.

FIG. 24 is a schematic showing of a system for treating cells, in accordance with an exemplary embodiment of the invention;

FIG. 25 is a flowchart of a method of treating cells, in accordance with an exemplary embodiment of the invention.

FIG. 26 is a flowchart of a method for treating blood cells, in accordance with an exemplary embodiment of the invention.

FIG. 27 is a schematic diagram of a system for treating blood cells, in accordance with an exemplary embodiment of the invention.

FIG. 28 is a schematic showing of an apheresis device for extracting blood components (e.g., plasma, erythrocytes, leucocytes, platelets and/or stem cells), usable in association with some embodiments of the invention; and

FIG. 29 is a schematic showing of a membrane hollow fiber reactor which can be used in association with some embodiments of the invention.

FIG. 30 is a flowchart of a method of electrical field application, in accordance with an exemplary embodiment of the invention.

FIG. 31A is a chart showing a proton induced uptake of dextran-FITC as a function of pH in erythrocytes, in accordance with some embodiments of the invention. As shown, PIU in erythrocytes depends on external pH in a constant rate. RBCs were exposed to different external pH in the presence of 70 kD dextran-FITC (10 μM) for a period of 5 minutes. Flow cytometry analyses include FITC intensity geometrical mean±SD presented as fold induction relative to background. n=9 in 3 independent experiments. This can be used, in some embodiments of the invention, to assess effect of pH on uptake in erythrocytes.

FIG. 31B is a chart showing a proton induced uptake of dextran-FITC as a function of time in erythrocytes, in accordance with some embodiments of the invention. RBCs were exposed to external pH 5.4 for time durations of 10, 15, 30, 45 and 60 minutes. Pulse label of 70 kD dextran-FITC (10 μM) was added to the cells for the last 10 minutes of every exposure. Flow cytometry analyses in terms of geometrical mean±SD are presented as fold induction relative the fluorescent intensity in the first group (10 min). No statistical difference is found in PIU rate for the different groups (p >0.05 by one way ANOVA, n=9 in 3 independent experiments). This can be used, in some embodiments of the invention, to assess effect of pH and time on uptake in erythrocytes.

FIG. 32A is a chart showing the kinetics of proton induced uptake of dextran-FITC by erythrocytes, in accordance with some embodiments of the invention. RBCs were exposed to external pH 5.4 for durations of 5, 10, 20, 30, 45 and 60 minutes in the presence of 70 kD dextran-FITC (10 μM). Flow cytometry analyses include FITC intensity geometrical mean±SD presented as fold induction relative to background. n=9 in 3 independent experiments. This can be used, in some embodiments of the invention, to assess effect of pH and time on uptake in erythrocytes.

FIG. 32B is a chart showing the kinetics of efflux of dextran-FITC from erythrocytes, in accordance with some embodiments of the invention. RBCs were pre-exposed to external pH 5.4 in the presence of 70 kD dextran-FITC (10 μM) for 10 minutes. The cells were washed and divided into 2 groups, suspended in fresh solution of either pH 7.4 or pH 5.4. Cells from each group were incubated for durations of 10, 20, 30, 45 and 60 minutes, before being analyzed by flow cytometry. Results given in terms of geometrical mean±SD are presented as fold induction relative to cells analyzed immediately following exposure to low pH. n=8 in 2 independent experiments. This can be used, in some embodiments of the invention, to assess effect of pH and time on uptake in erythrocytes.

FIG. 33 is a chart showing repeated PIU of dextran-FITC by RBCs, in accordance with some embodiments of the invention. One treatment cycle includes the RBCs exposure to low pH solution in the presence of dextran-FITC, thereafter subjecting them to washing with buffer solution (e.g. PBS) and suspending in PBS-G for 10 minutes. Following treatment the cells were washed in PBS-G and analyzed by flow cytometry. Results are presented as geometrical mean of FITC intensity±SD for 50,000 cells per sample. This can be used, in some embodiments of the invention, to assess effect of repeated PIU protocols, for example, in erythrocytes.

FIG. 34 is a chart showing the dependence of PIU on extracellular pH, in accordance with some embodiments of the invention. TK6 cells were exposed to solutions of different pH, in the presence of dextran-FITC for a period of 5 minutes. Following the cells were washed, harvested and analyzed by flow cytometry. Uptake, based on flow cytometry, is plotted as function of the external pH from 3 independent experiments. Results are presented as folds induction of the geometrical mean±SD (10,000 cells/sample) of cells exposed to low pH relative to control cells exposed to normal pH. This can be used, in some embodiments of the invention, to assess effect of pH on uptake.

FIG. 35 is a chart showing the PIU rate in TK6, in accordance with some embodiments of the invention. TK6 cells were exposed to low pH solution for increasing periods of time (0 to 60 minutes), followed by additional 10 minute exposure to low pH in the presence of dextran-FITC, followed by washing step and flow cytometry analysis. Results are presented as folds induction of the geometrical mean±SD (10,000 cells/sample) of cells exposed to low pH relative to control cells exposed to normal pH. This can be used, in some embodiments of the invention, to assess effect of pH and time on uptake.

FIG. 36 is a chart showing TK6 cells exposed to PIU for extended periods of time, in accordance with some embodiments of the invention. TK6 cells were exposed to low pH solution in the presence of dextran-FITC for up to 60 minutes, then washed and analyzed by flow cytometry. Results are presented as folds induction of the geometrical mean±SD (10,000 cells/sample) of cells exposed in the presence of dextran-FITC relative to control cells exposed without dextran-FITC. This can be used, in some embodiments of the invention, to assess effect of pH and time on uptake.

### DESCRIPTION OF EMBODIMENTS OF THE INVENTION

The present invention, in some embodiments thereof, relates to controlling uptake of materials by cells and, more particularly, but not exclusively, to controlling uptake by pH modification.

A broad aspect of some embodiments of the invention relates to controlling uptake of formulations into a cell by modifying a local chemical environment of the cell, for example, pH, in a manner which encourages vesiculation and/or invagination. In an exemplary embodiment of the invention, the modification is that of decreasing pH. Optionally, the decrease is not to levels so low as to destroy too many of the cells. In an exemplary embodiment of the invention, the modification does not grossly affect the volume of the cell or its integrity, as might be by poration methods and osmolarity changes. In an exemplary embodiment of the invention, the cells are substantially unharmed by the act of vesiculation (however, materials which enter may be selected to be of a toxic nature). In an exemplary embodiment of the invention, the uptake operates on uncharged molecules.

In an exemplary embodiment of the invention, the uptake is defined as a ration to uptake in a cell in a neutral environment. For example, in some embodiments, uptake is a factor of 5, 10, 20, 30, 50, 100 or more of baseline uptake. It is noted that in some cases uptake is modulated by expelling by cells of uptaken materials, but in general, uptake rate can be made higher than expelling rate, until rather high intracellular concentrations are reached.

In an exemplary embodiment of the invention, the uptake does not utilize electrophoresis or transport of charged molecules as a substantial component. As noted below, however, charged molecules may be better placed for transport, when it occurs.

In an exemplary embodiment of the invention, the uptake rate and/or amount and/or temporal profile (e.g., using repetitions) is calculated based on a formula in which uptake rate is linearly dependent on time, concentration and temperature and exponentially dependent on the pH. Different materials may have different calibration values. In addition to influx, there is often efflux, which generally depends logarithmically on the intracellular concentration of the materials. Additional modifiers, for example, as described herein, include membrane stiffness, material affinity for the membrane and membrane potential. It is also noted that uptake caused by chemical means and uptake caused by application of an electric field can be treated equivalently, in accordance with some embodiments of the invention. It is believed that the results shown herein are sufficient for estimating parameters for causing uptake of a wide variety of materials in a wide variety of cells. It is also noted that the actual uptake parameters and/or additional material used may be limited by consideration of cell viability. In addition, it is noted that while the experiments generally show results at discrete values of parameters, one of ordinary skill can interpret expected results and, where the graphs are not near saturation, extrapolate as well.

In an exemplary embodiment of the invention, local elevation of hydrogen ions (protons) is provided by one or more of the following methods:
1. Direct addition in free form, for example adding soluble formulations of acidic nature. Such formulations may be added locally to the cells, or, for example, systemically, for example, by IV or by direct injection.
2. Release from solid, semi-solid or liquid substances, for example from mesoporous ceramics, hydrogels or polymers such as Eudragit L100-55.
3. Using a proton exchange membrane (PEM) that separates hydrogen to ions to locally generate hydrogen ions, for example, in an implanted device, with the membrane adjacent cells to be treated and optionally adjacent a source of material to be taken up.
4. Chemical cleaving of molecules, for example hydrolysis of water.
5. Electrical cleaving of molecules, for example electrolysis of water, in particular by applying an anodic current (anode near the cells).
6. Extracellular acidification by acidifying the whole body, for example, by increasing the CO₂ content of inhaled air, medication, or certain types of activities or ventilations. Such acidification may be used together with electrically-mediated uptake.
7. Extracellular acidification by acidifying a restricted part of the body such as organ or part of an organ, for example, by confining the blood circulation to that organ, by increasing metabolic activity (e.g., and lactic acid formation) therein and/or by blocking lactic acid neutralization or other metabolic activity that reduces acidification.
8. In vitro treatment of cells.

In an exemplary embodiment of the invention, an anodic current is applied to the cells to provide or enhance said encouraging chemical environment. In an exemplary embodiment of the invention, the current is in the range of 1 mA to 200 mA when employing platinum electrodes. Optionally or alternatively, the duration of application is between 1 second and 60 minutes, for example, 15 minutes. For example exposing cells suspended in 0.5 ml, PBS to electric pulse train of 100 mA/cm2 for 1 min will reduce pH level to 3.5. In an exemplary embodiment of the invention, the current density used is selected to be below a level which would directly damage cells, such as by poration, and optionally lower than needed for effective electrophoresis.

In an exemplary embodiment of the invention, however, provision of protons by chemical means, from a chemical source, is used to lower pH. In some embodiments, no electrical fields are externally applied.

In an exemplary embodiment of the invention, the uptake is caused by chemical interactions and not significantly mediated by biochemical interactions. For example, membrane channel and gate proteins and/or other transmembrane proteins do not act. It is noted that the mechanical stiffness and/or other properties of the membrane may affect uptake and these may be caused by mechanical behavior of proteins.

In an exemplary embodiment of the invention, a plurality of environment modification methods are used together. For example, this may be used to improve targeting. In one example, a first reduction in pH is provided via chemical acidification and a second reduction is provided using anodal current. For example, this allows a first acidification (and uptake) to be provided to an area including a tumor, with electrical current being used to fine tune uptake. Optionally, methods not related to acidification are used together with pH-based vesiculation, for example, sonoporation or electroporation or chemically or bio-chemically includes active vesiculation.

In an exemplary embodiment of the invention, the pH modification is timed and set to levels which will kill fewer than, for example, 50%, 25%, 10% or 2% of the cells being treated. In some embodiments, the number of cells which are allowed to die depends, for example, on whether the treatment is in-vivo and/or on a solid mass of cells, as in such cases, as opposed to ex-vivo on separate cells, it may be desirable to avoided significant tissue destruction. In separated and/or ex-vivo cell cultures, it may be convenient to apply harsher conditions and separate out damaged cells.

Optionally, the uptake is provided in pulses, for example, with one or both of the pH and material to be uptaken provided in pulses and/or otherwise modified over time. Optionally, this is done in response to a feedback signal, for example, a pH sensor, which indicates the actual pH, from which uptake can be estimated. If the uptake is too low, application may be repeated. Optionally or alternatively, in order to reduce damage to cells and/or allow for an effect of an uptaken material (e.g., before a next dose or before applying a second material), a delay is provided between pulses. In some cases, a first material (e.g., siRNA) is provided to prevent the cell form being able to react to a second treatment, such as a toxin which is normally pumped out by enzymatic pumps. A delay during which the enzyme is degraded may be provided. Optionally or alternatively, a delay until the DNA is blocked is provided, after which the enzyme is directly deactivated by chemical means. In another example, a different treatment, such as heating or radiation or chemical treatments is applied after some cells are made more sensitive by uptake and/or after some cells are protected by uptake. In some example, the opposite is provided—uptake is enhanced after a treatment, for example, to enhance the effect of the treatment or to prevent reaction thereto. In a specific example, a material which reduces expelling of the uptaken material is provided to improve the effect of uptake. In another example, a material which modifies the cytoskeleton is provided to increase or decrease uptake.

In an exemplary embodiment of the invention, the local environment is modified to include an increase in free radical precursors.

In an exemplary embodiment of the invention, the cells are treated while inside a body, for example, using implanted or inserted electrodes and/or sources of formulations.

In an alternative embodiment of the invention, the cells are treated while outside the body. In one example, cells or tissue are extracted from a body by one or more of surgery, biopsy, dialysis and/or aphaeresis. The cells or tissue are then optionally exposed to high concentration of hydrogen ions with associated formulations and returned to the body. For example, this method may be used for micro-organ gene transfer, gene silencing or treatment of blood cells (e.g., red or white blood cells).

Various cell types can be used. For example, the cell can be a eukaroyotic or a prokaryotic cell. The cell can be an isolated cell or a cell which forms a part of a tissue. In ex-vivo or in-vitro applications the cell can be a primary cell or a cell-line.

Accordingly, the cell can be an adherent cell or a cell cultured in suspension. The cell can be genetically modified (e.g., using standard modes of transformation/transfection) or a naïve (i.e., non-genetically modified) cell. The cell can be a terminally differentiated cell or a stem cell.

Eukaryotic cells include, but are not limited to, plant cells, insect cells, yeast and mammalian cells.

Examples of differentiated cells include, but are not limited to, liver cells, cardiac cells, muscle cell, fat cell, neural cells, cone cell, cartilage cell, connective tissue cell, blood cell, secretory cell (e.g., islet cell), skin cell, hair/hair follicle cell, reproductive-system cell.

Examples of stem cells include, but are not limited to, pluripotent stem cells (e.g., embryonic stem cells, induced pluripotent stem cells), progenitor cells, mesenchymal stem cells (e.g., bone marrow, placenta or adiopose tissue) and neural stem cells.

The cells can be normal unaffected cells or diseased pathogenic cells. Examples of pathogenic cells include bacterially infected cells, viral infected cell, a pre-malignant cell, a malignant cell, a cancer cell and an abnormally activated immune cell

In an exemplary embodiment of the invention, pretreated cells are packaged and distributed, for example, pre-treated red blood cells or stem cells. In another example, blood is removed from a person, optionally separated into constituents, treated and optionally stored before being reintroduced into the body.

In an exemplary embodiment of the invention, a kit is provided including uptake materials, optionally uptake promoting materials. Optionally, the kit includes a filter media and/or a reactor chamber where uptake is to take place. Optionally, the reactor is preloaded with acidification materials and/or electric field causing elements.

Optionally, exposure ex-vivo is used for biotechnological or research purposes, for example genetic transfection of proteins or growth factors. In one example, bacteria, plants, or other (e.g., animal, yeast, fungal or single) cells are transfected with vectors, by exposing them to acid or by applying an anodal current. For example this will be carried out in a device where cells are suspended in physiological solution (or living on a substrate) which flows through a porous dialysis hollow fiber with a cutoff appropriate for the drug or particle to be introduced into these cells. The hollow fiber path transverse one chamber which contain the designated drug or nanoparticles in the immediate vicinity of the anode. Alternatively the chamber may contain an acidic solution in the range of pH 4 to pH 6 containing the drug or the nanoparticles. Following the uptake in the anodic of low pH chamber the fiber go through another chamber containing a physiological neutral solution (pH=7.2-7.4).

In an exemplary embodiment of the invention, the cells (in-vivo or in-vitro) are exposed to one or more formulations (or other agents) comprising one or more of the following:
a. Nucleic acid sequences of DNA, which are optionally used for introducing cells with new properties and functions, correcting resident mutations and/or silencing existing functions. Optionally, the sequences are included in, for example plasmids and/or vectors (e.g., viral vectors). For example the introduction of the gene for insulin into type 1 diabetics or silencing the genes responsible for the production of protein that generates auto-immune reaction.
b. Nucleic acid sequences of RNA, which are optionally used for interfering with protein expression and/or cell functioning, for example anti-sense RNA and siRNA (e.g., that silence the production of defective receptors).
c. Active molecules (e.g., organic or biologic molecules) such as inhibitors, blockers or promoters that interfere with cell functions. Exemplary molecules include, for example, chemotherapy, toxins, peptides, proteins, antibodies and/or fatty acids. For example the anti-oxidants Dihydroascorbate can prevent pro-apoptotic activity. Toxic agents can be used to selectively kill a population of cells of interest, such as pathogenic cells e.g., cancer cells. The use of toxins is also valuable in agriculture and is included herein as pesticides killing a parasite of interest.
d. Inorganic formulations that interact with cell functions such as, for example, formulations that interact with ionophores, enzymes, catalytic domains and/or respiration chains. For example a slow release formulation of Galium (Ga), a competitive ion to Fe that inhibits iron dependent enzymatic activity.
e. Formulations which target therapeutic agents to specific sites in a cell, for example, by incorporating a targeting agent and the therapeutic agent into an endocytosis-created vesicle; binary compositions and formulations that assist targeting particular cells and/or maintaining desired concentrations within or without a cell. Optionally, a targeting agent includes a ligand for receptors and/or one or more antibodies. Optionally or alternatively, the formulation includes molecules possessing an appropriate size for enhance permeability retention, e.g., nanoparticles associated with chemotherapies, possessing sizes in the range of 5-150 nm. These size range is appropriate for passive targeting through enhanced permeability retention (EPR). Optionally or alternatively, the targeting agent enhances local accumulation by physical forces, for example, using such as magnetic beads. For instance nano-size magnetic beads coated with polymers, which are associated with a drug, may be used. These beads, once formulated in a microbead scale, can be localized by strong external magnetic fields at the required site for drug release. The release can be initiated by temperature elevation (e.g., via exposing the body area where the magnetic beads concentrate to external AC electric or RF field) consequently releasing the drug and the hydrogen ions.
f. Any of the above attached to nano particles
g. Any of the above conjugated with or otherwise linked to a material that attaches or approximates a cell membrane. For example conjugation with charged polymers (like dextran) that result in greater adsorption to the cell membrane.
h. In an example of targeted treatment of malaria whereby the parasite residing in the red blood cells is eradicated using the present teachings and the anti malaria agent can include standard medications (e.g., quinacrine, chloroquine, primaquine, mefloquine (Lariam), doxycycline (available generically), and the combination of atovaquone and proguanil hydrochloride (Malarone)), thereby potentially significantly lowering side effects associated with anti malaria medications;

In an exemplary embodiment of the invention, the formulation and/or desired target concentration in a cell and/or targeting agent are selected to have a desired therapeutic effect. Optionally or alternatively, the timing of delivery and/or duration of maintaining desired levels are selected for a desired therapeutic effect. In an exemplary embodiment of the invention, uptake is by multiple exposure, for example, multiple exposure to low pH produced either electrically or chemically, may be more effective and/or decrease viability less than long continuous exposure to a higher pH. In one example multiple use of 1 minute exposures to pH 4, with interpulse duration of 5-10 minutes, may constitute an exposure profile. Different exposure lengths and/or may be selected (for example based on cell sensitivity). Different interpulse durations may be selected, for example, based on recovery time of cells. Other exemplary pulse lengths include 10 seconds, 30 seconds, 2 minutes, 5 minutes, 10 minutes and shorter, intermediate or longer periods. Other exemplary interpulse durations include 2 minutes, 20 minutes, 45 minutes 1 hour and shorter, intermediate or longer periods. Exemplary pH levels used for uptake for this and other embodiments include 3, 3.5, 4, 4.5, 4.8, 5, 5, 1, 5.2, 5.4 and smaller, intermediate or greater pH values.

In an exemplary embodiment of the invention, the formulation is selected so it easily fits in typical vesicle sizes, for example, between 10 and 300 nanometers, for example, between 10 and 200, 10-100, for example 50 nanometers. For example, formulations/particles may be selected to have a size smaller that 200 nm, 100 nm, 50 nm, 30 nm or intermediate sizes, in their largest dimension.

In an exemplary embodiment of the invention, the formulation concentration is selected or controlled according to a desired uptake rate. Optionally, the formulation tends to adsorb to cell membrane, modifying the concentrating effect on uptake rate. In an exemplary embodiment of the invention, however, the formulation does not significantly adsorb to the membrane.

In an exemplary embodiment of the invention, the expected uptake rate of a material is calculated taking into account the ability of the applied material to adsorb to and/or approach the cell membrane, as the contents of a vesicle are appear to have a greater concentration of such adsorbing and approaching materials. Optionally, a material to be uptaken is modified so it adsorbs. In an exemplary embodiment of the invention, the materials are adsorbed to charged polymers and/or membranes with hydrophobic zone.

In an exemplary embodiment of the invention, the extracellular concentration of formulation, delivery of formulation, constitution of the formulation, duration of pH modification and/or repetition thereof are controlled to achieve an intra-cellular concentration within a desired range for example, within a factor of 10, 5, 4 or 2 (or intermediate or greater or smaller factors). Optionally, one or more of the following are modified to achieve these various uptake increases: extracellular concentrations, exposure time, temperature, pH.

In an exemplary embodiment of the invention, the parameters of acidification and/or electric field application are selected to have a desired increase in uptake, for example, a factor of 2, 4, 6, 0, 20, 30, 50, 100, or greater, smaller or intermediate factors. Optionally or alternatively, for some cells, the parameters of chemical environment, are selected to reduce uptake relative to that expected based on pH and/or electrification, for example, by a factor of 2, 4, 10, 20 or smaller, intermediate or greater factors.

In some cases, tissue is treated where a factor of uptake between different cell types is set to be (e.g., by selecting a certain acidification level), for example, a factor of 2, 4, 6, 10, 20 or smaller, intermediate or larger factors.

In an exemplary embodiment of the invention, the formulation includes an expulsion control or metabolizing material which modifies, for example, reduces or increases a rate of expelling or metabolizing or inactivating or activating of the formulation from/in cells. In one example or achieving differentiated intracellular levels, rate is reduced in the treated cells. In another example, rate is increased in all cells, but intake is accelerated only in treated cells. Exemplary expulsion inhibiting formulations include inhibitors of transporters such as MDRs such as cyclosporine or verapamil or inhibitors of exocytosis.

In an exemplary embodiment of the invention, rather than externally providing a formulation to be taken-up by cells, that formulation is naturally present, for example, being in inter-cellular fluid (e.g., a hormone or a nutrient for example glucose), its uptake in treated cells controlled using methods as described herein.

An aspect of some embodiments of the invention relates to a method of cell modification whereby a formulation, selected to be differentially and meaningfully taken up by some cells of the body, based on an expected pH of said portion, is used. In an exemplary embodiment of the invention, the cells are outside the body while being treated, for example, as blood. Optionally or alternatively, the cells are inside the body during treatment.

In an exemplary embodiment of the invention, the expected pH is controlled, for example, using an electric current or chemical application. Optionally or alternatively, the expected pH is at least partly due to the tissue behavior, for example, being cancerous tissue, where pH level drops to levels of 6.2-6.8.

In an exemplary embodiment of the invention, the cells to be treated are selected from a tumor, an ulcer, lymphocytes, erythrocytes, blood vessels, muscles and/or any part in the body accessible by a fluid port and/or electric fields, for example, using a needle, a catheter and/or an endoscope, for example, by local application to cartilage in the joints, to intestinal infection site or to a cancerous tissue.

In an exemplary embodiment of the invention, increased hydrogen ion concentration and/or medication are provided using an invasive device, for example, using a needle, cannula or catheter to deliver materials. Optionally or alternatively, such devices are used to deliver one or more electrodes for applying an electric field. Optionally, a cathode is provided at a remote location, for example, outside the body.

In an exemplary embodiment of the invention, increased hydrogen ion concentration and/or medication are provided using implanted means, for example, one or more of:

(a) Local release from implanted micro-fluidic devices such as electro-mechanic pumps or osmotic pressure pumps;

(b) Application of anodic current, for example by implanted electrodes;

(c) Induction of anodic current between the poles of implanted conductive electrodes;

(d) Enzymatic or chemical degradation of a hydrogen ion containing unit, such as a lattice, a matrix, polymers, particles and/or scaffolds, which are implanted (optionally within a device) in the treatment area; and

(e) Lattice, matrix, polymers, particles, scaffolds and/or hydrogels which release hydrogen ions. Said release can be, for example, slow, fast, pulsed and/or controlled. In some examples, control is by local triggering events or by external signals by the use of temperature sensitive Pluronic gel, electro sensitive polymethylacrylate, light sensitive leuco derivate polymers and/or pressure sensitive poly(N-isopropylacrylamide).

An aspect of some embodiments of the invention relates to taking electro-chemical effects in to account when applying an electrical therapy. In an exemplary embodiment of the invention, a therapy which includes proton-mediated membrane modification is modified by selectively increasing or decreasing an amount of buffering and/or a local pH. Optionally or alternatively, the field to be applied is modified to take into account an expected buffering ability, pH and/or to support targeting of cells. In an exemplary embodiment of the invention, the following formula is used to convert between pH effects and current effects: current*time/buffering=pH effect.

An aspect of some embodiments of the invention relates to uptake of materials into red blood cells (or in other cells). In an exemplary embodiment of the invention, the process used has a high yield, for example, over 50%, over 75%, over 90% or intermediate yields. Optionally or alternatively, the process has a very low cell damage rate, for example, less than 10%, less than 5%, or less than 1% red blood cell damage by the process. Optionally or alternatively, the process allows the insertion of high weight molecules, for example, with a molecular weight of above 70 kD. In an exemplary embodiment of the invention, the insertion is of molecules having a diameter of less than that of the created vesicles or invaginations, for example, less than 80%, less than 50%, less than 30%. Optionally, the molecules have a diameter of more than 10%, more than 20% or more than 30% of the vesicles. Optionally, the molecules are treated (e.g., with a proton sponge) so that they have a smaller maximum diameter.

In an exemplary embodiment of the invention, the low damage caused to cells allows the serial uptake of multiple materials, for example, materials which are incompatible in solution, due to reactions between them. Optionally, at least two, at least 3, at least 4 materials are added to a cell, for example a red blood cell, by sequential acts of uptake using pH-mediated methods as described herein.

### Discussion of Supporting Experimental Results

A plurality of experiments was carried out and is discussed below. It should be noted that the scope of some embodiments of the invention need not be necessarily be limited by the theoretical discussion below in which various hypotheses are suggested. Some of the experiments were carried out to differentiate between alternative hypotheses. Others, to better characterize the parameters of uptake, for example, so as to apply various embodiments as describe herein. In particular, it is believed that based on the results provided herein, a practitioner can calculate application parameters which will result in a desired uptake amount and/or treatment effect. It is also noted that the experiments do not show every possible parameter value and combination. However it is believed that the ranges taught herein are clearly support by the experiments.

It has been shown that exposure of cells to a short train of pulsed low electric fields leads to a stimulated uptake of different fluid phase and adsorptive fluorescent probes via endocytic pathways (8). The exposure to the electric fields resulted in an alteration of cell surface, leading to elevated adsorption of macromolecules (bovine serum albumin (BSA), dextran, and DNA) to it with consequent enhanced uptake (9). This surface alteration, attributed to the electrophoretic segregation of charged components in the outer leaflet of the cell membrane, was suggested to be responsible both for enhanced adsorption and stimulated uptake, via changes of the membrane elastic properties that enhance budding and fission processes (9).

There is apparently little or no evidence in the body of scientific literature for the induced penetration of non-pathogenic molecules, let alone fluid phase molecules such as dextran following extracellular acidification. Acid induced uptake of naïve proteins such as lactalbumin, ovalbumin and horseradish peroxidase was demonstrated to be pH dependent in fibroblast monolayer following 30 minutes incubation in acidic solution, and suggested to follow a non-endocytotic pathway, since it did not show sensitivity to temperature or ATP depletion (10).

There is some work that describes the relation between acidity and membrane penetration by viral and bacterial proteins, mostly in the context of endosomal escape. Briefly, Viral fusion proteins contain a hydrophobic segment referred to as the “fusion peptide,” which, in most cases, is initially buried within the pre-fusion form; however, once fusion is triggered, it is exposed and can associate with the membrane of the host cell. In this transition phase, the protein is anchored in the viral envelope and the host cell membrane simultaneously, and further conformational changes drive the two membranes to fuse (11-17). One reason for choosing dextran, a glucose polymer, as an uptake probe in some experiments herein, is that it is unlikely that the acid induced entry to the cells is carried along the same mechanism as those of viral proteins.

In brief summary of the experiments, it has been discovered that the exposure of cells to external low pH at near physiological values, result in an augmented uptake of fluid-phase dextran. The low-pH derived uptake (LpHDU), also termed PIU (Proton induced uptake) takes place at a fairly constant rate; however the accumulation of dextran in the cell is obscured by the cell's ability to actively expel dextran out. The result of these two contradicting processes is the LpHDU saturation curve. In a practical application, as described herein, for example, a practitioner may control one or both of uptake and expulsion.

Dextran was used as a substitute for other formulation which may be taken up and which may have different kinetic behavior, and as a convenient, non-toxic material to assay cellular behavior, whereas other formulations may be toxic (e.g., chemotherapy).

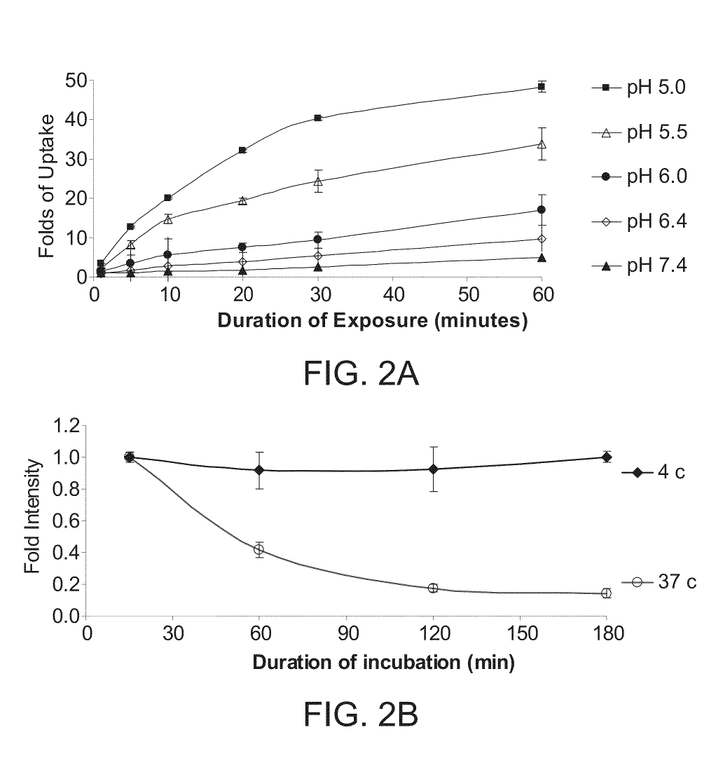

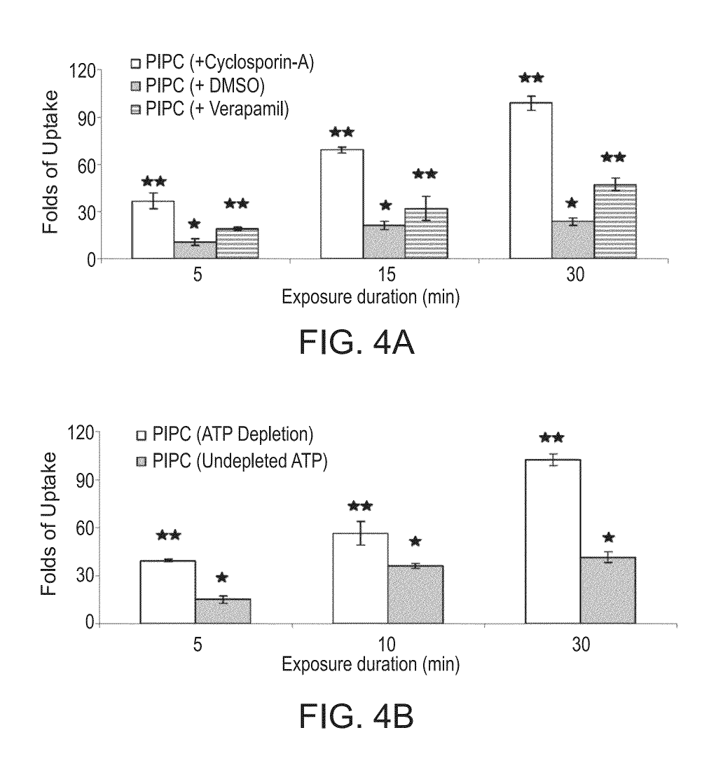

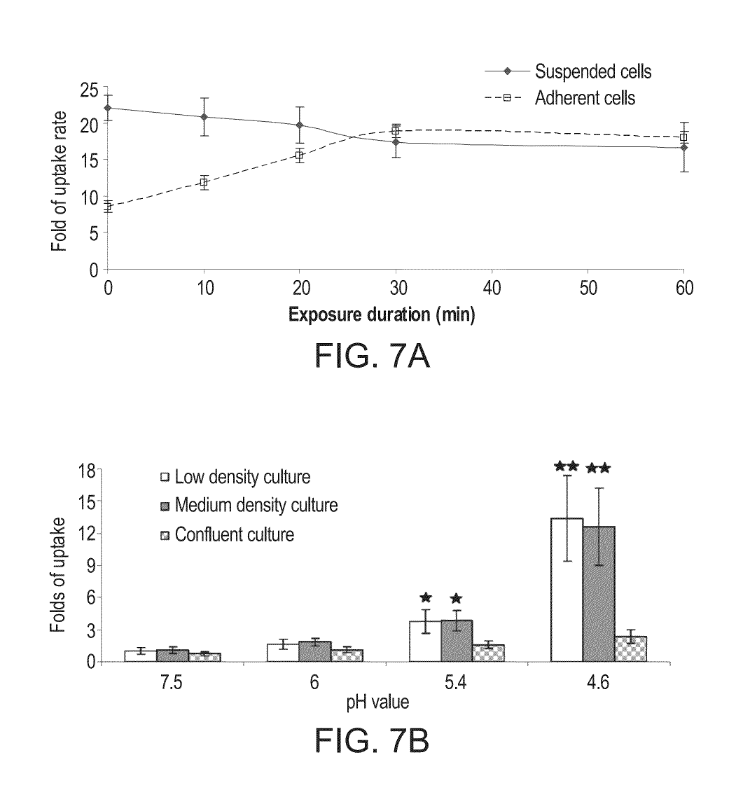

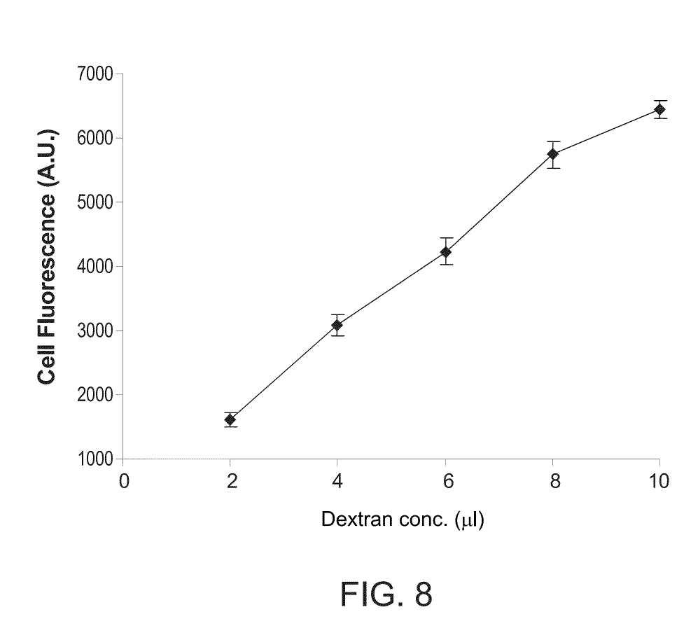

The kinetics of PIU mediated uptake of dextran by cells emerges as having a constant rate, reflected in pulse labeling studies (FIG. 7A). However, the kinetics of dextran accumulation in the cells could take the form of saturation curve under increasing exposure durations. This saturation curve becomes more pronounced as the external pH is lowered, gradually shifting from a linear correlation at pH values close to pH 7.4, to a logarithmic shape at pH 5 (FIG. 2A). This apparent difference is studied by applying inhibitors of cellular efflux ATP binding cassette (ABC) pumps using cyclosporine-A or verapamil, or when the entire cellular ATP pool is depleted. Under these conditions, the cellular accumulation of dextrans as function of time shows a linear relationship (FIG. 4A). Thus the intracellular concentration of dextran represents the balance between its afflux and efflux. The efflux of dextran was studied by measuring the time dependent decrease of intracellular dextran concentration that follows the cells exposure to low external pH. The observed decline rate in cellular fluorescence features an exponential shape at 37° C. (FIG. 2B), suggesting the efflux rate to be a metabolically driven process since it is abolished at 4° C. The constant prevalence of PIU is expected to be followed by reduction in the cell area/volume ratio, consequently increasing the membrane tension and bending rigidity. The finding that PIU rate is constant for at least one hour of exposure, suggests that no reduction in plasma membrane area occurs. Therefore membrane vesicles or invaginations (e.g., with a strong curvature, which encourages leakage into cells) are probably short lived and efficiently fuse back to replenish the plasma membrane area.

In an exemplary embodiment of the invention, LpHU is practiced for relatively long periods, independently of cell energy levels and/or at pH values which do not significantly damage cells.

The results suggest that there are actually at least two processes that govern intracellular loading of dextran during the exposure to low pH; one is the inward uptake of fluid phase dextran and the other one is the outward expel of that dextran. Based on the findings, it is assumed that the removal of dextran-FITC from the cell is performed by active, energy consuming, mechanisms involving exocytosis or MDR based processes. Optionally, such processes are blocked or enhanced, as desired, to affect intra-cellular concentration.

The acid induced influx of dextran into the cell is optionally represented by equation 1. Since dextran concentration in the external suspension medium (S₀) can be regarded as constant during the uptake process (due to extremely high suspension volume/cell volume ratio), plotting the internal dextran concentration [Sᵢ] vs. t will yield a linear plot with kᵢₙ slope, as demonstrated in FIG. 4A.

$$\frac{d [ {S}_{i} ]}{d t} = {k}_{in} [ {S}_{0} ] \qquad ( Eq . 1 )$$

The efflux of dextran from the cell described in FIG. 2 is an energy consuming process, as demonstrated in FIG. 4. It is assumed it could be described by the Michaelis-Menten kinetic (equation 2) and hence one can use equation 3 to describe its characteristic Km, with T standing for the transporter, Sₜ for the transported dextran and Kₜ for the transporting kinetic factor.

$$\begin{matrix}{S}_{i} + T {⇔}_{k - 1}^{k 1} {S}_{i} T {}^{k 2} {S}_{t} + T & ( Eq . 2 ) \\ Km = \frac{{k}_{- 1} + {k}_{2}}{{k}_{1}} = \frac{[ {S}_{i} ] [ T ]}{[ {S}_{i} T ]} & ( Eq . 3 )\end{matrix}$$

Michaelis-Menten defines product formation (dextran transport in this case) as:

$$\frac{d [ {S}_{t} ]}{d t} = {k}_{2} [ {S}_{i} T ] = \frac{{k}_{2}}{Km} [ {S}_{i} ] [ T ] = {k}_{t} [ {S}_{i} ] [ T ] \qquad ( Eq . 4 )$$

Equation 4 is in agreement with the data presented in FIG. 2B indicating the efflux of dextran to be a second order ODE that yields a linear plot when 1/[Sᵢ] is plotted vs. time.

Optionally, the total accumulation of dextran in the cell is described by equation

$$\frac{d [ {S}_{i} ]}{d t} = {k}_{in} [ {S}_{0} ] - {k}_{t} [ {S}_{i} ] [ T ] \qquad ( Eq . 5 )$$

In order to estimate the kinetic profile of kᵢₙ at different pH values one can look at the initial reaction where efflux is still ineffective and the acid induced uptake is the only mechanism responsible for the intracellular dextran load.

[Sᵢ]ᵢₙᵢₜᵢₐₗ=kᵢₙ[S₀]Δt (Eq. 6)

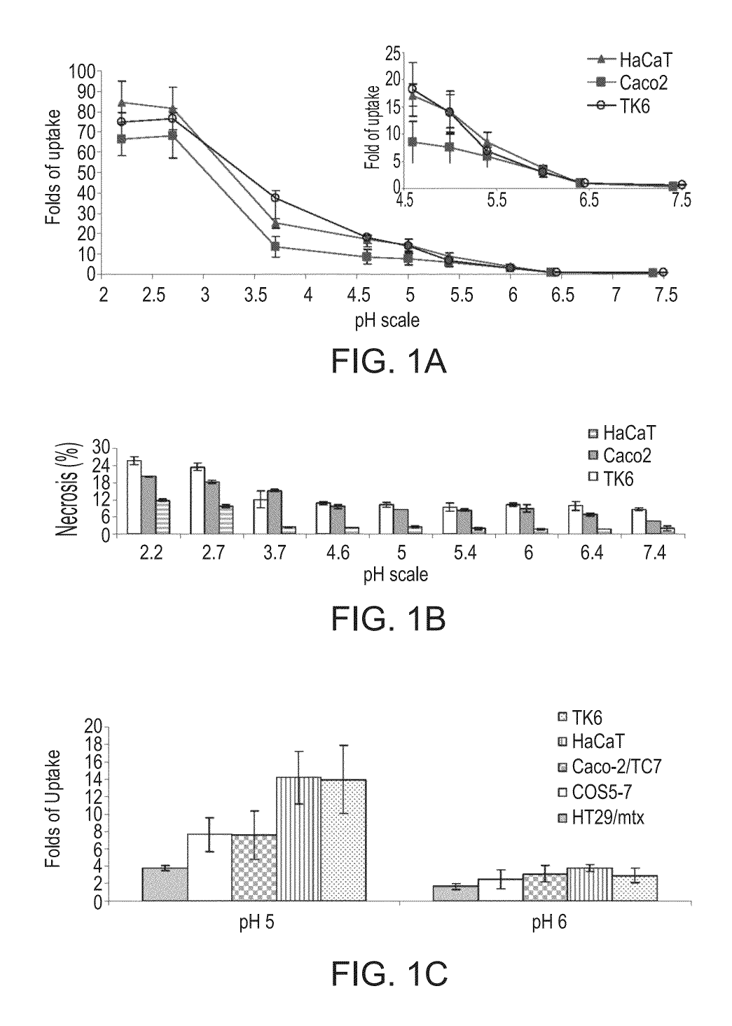

For equation 6, both external dextran concentration (S₀) and the initial exposure (Δt=60 seconds) are constant during experimental series, and [Si] can be substituted by kᵢₙ in the Y axes of FIG. 1. FIG. 1 is then used to portray the profile of uptake rate at different pH values, showing it to be exponential from about pH 6 to about pH 3, where it reaches its maximal value. The actual pH used may depend, for example, on allowed rate of cell death, cell sensitivity to pH and/or rate of intake desired.

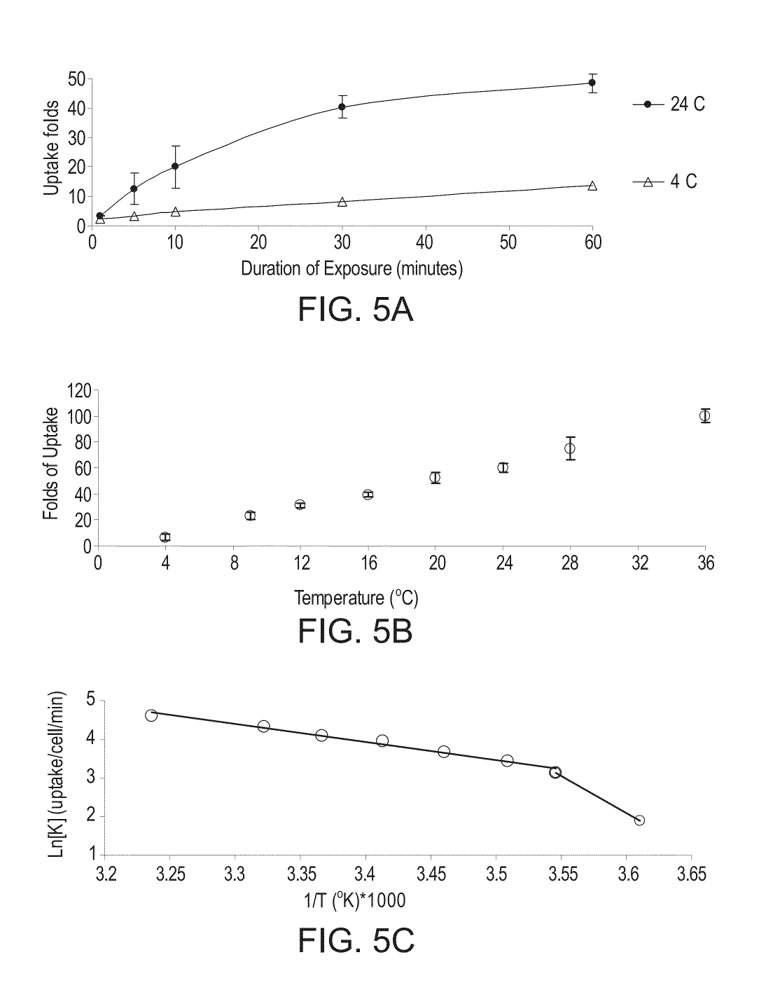

The possibility that cell exposure to external low pH activates the pathways underlying the constitutive endocytosis was explored, by using common inhibitory pharmacological procedures while monitoring the extent of PIU in cells under these conditions. The findings clearly show that subjecting the cells to a battery of conditions or agents that are known to inhibit the clathrin and caveolin mediated endocytosis or inhibit macropinocytosis, has no attenuating effect on the extent of PIU mediated uptake in cells. Moreover, PIU is accompanied by reduction of cytosolic pH, a condition known by itself to inhibit endocytosis (18). A further support for PIU independence of the classical endocytosis is reflected through the prevalence of PIU under low temperature and ATP deletion (FIGS. 4b and 5). The energy required to curve the membranes and the phosphorylated state of the proteins machinery involved, explains why maintaining long term endocytosis requires additional ATP hydrolysis as an energy source.

The experimental data indicates that the uptake event is accompanied by the formation vesicular bodies or intracellular membrane invaginations. It is shown, by electron micrographs, that gold nanoparticles are mostly aggregated in groups within the cell body or at cytoplasmatic extensions. Some nanoparticles are found dispersed within the cytosol, which could be the result of vesicular escape. Optionally, such escape is used for when targeting parts of the cytosol. Optionally, the uptake formulation is formulated to affect vesicle breakdown rate, for example, making it slower or faster than normal, for example, to be 1-10 minutes, 30-50 minutes, 1-2 hours or 4-6 hours, or intermediate durations.

Cell viability was tested under moderately low pH. Initially, a fraction of the cells becomes necrotic, but those who survive remain viable. Such response could be attributed to variability in cell functionality or integrity, suggesting that most of the cells are capable of enduring the higher hydrogen ion concentration. In contrast, at the lower pH 3.5, cells are damaged continuously at a near constant pace probably due to their inability to withstand such extreme conditions and preserve viable conditions. Optionally, some processes, such as genetic transfection of bacterial or other cells of substantially unlimited availability, may be carried out at low pH with damaged cells being removed or ignored in the final product. Optionally, when treating body tissue which is to be returned to the body, lower rates of cell death are desired. In an exemplary embodiment of the invention, cells to be transfected are mixed with a formulation and passed through an anodic chamber (described below), with the time of passage and applied fields determining controlling the uptake. The cells are optionally filtered out and the formulation optionally reused.

Cell metabolic activity following exposure to a range of low pH levels was tested. Exposure was conducted in 96 well-plates. Cell ware incubated up to 2h in pH calibrated HBSS, thereafter their metabolic activity analyzed by the Alamar-blue assay. Actual pH values were measured for each well with a pH electrode.

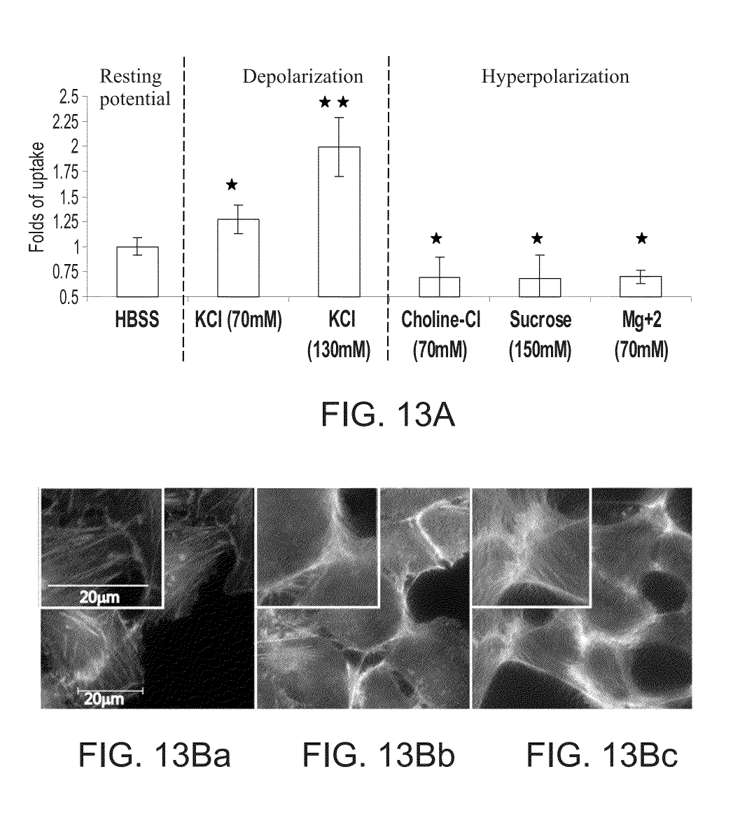

The cell's cross-membrane potential difference was found to have a significant affect on the rate of PIU (FIG. 13). The data reveals that de-polarization of the cross-membrane potential is accompanied by two folds increase in PIU relative to cells with unmodified resting potential. The opposite effect of hyper-polarization of the cell's resting potential resulted in a 25% decrease in PIU. In some embodiments of the invention, this cross-membrane potential will be modified, for example, by changing the local ion concentrations in order to have a desired effect on uptake.

Similarly, pH clumping with nigericin did not reduce the extent of the measured uptake, suggesting that PIU is independent of ΔpH. In another test, reduction in the negativity of the cell zeta potential by enzymatic digestion of the cell glycocalyx coat or reduction of the membrane surface debye length by increasing the ionic strength, both had non significant effect on PIU. These insensitivities to trans-membrane ΔpH and Z potential lead to a hypothesis that the driving force for the acid induced uptake results from an increase in the plasma membrane charge asymmetry.

It is hypothesized that the electric field operates at least in part by changing pH, rather than (only) by other electrical effects.

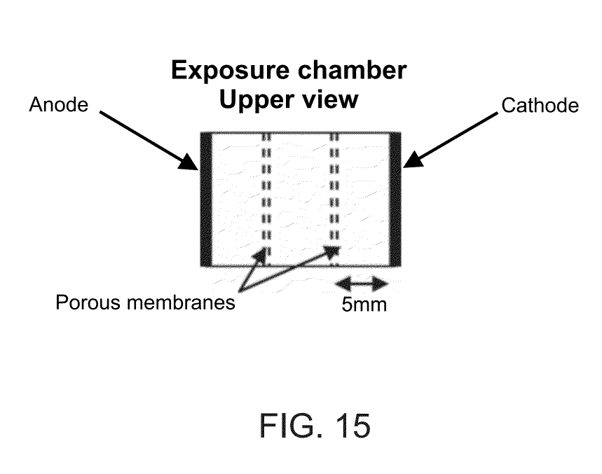

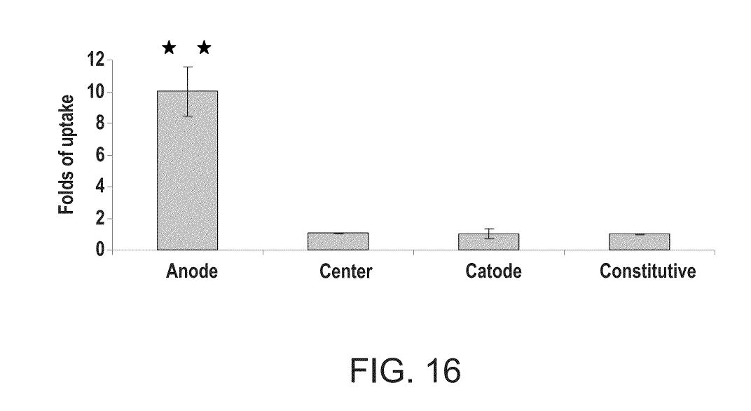

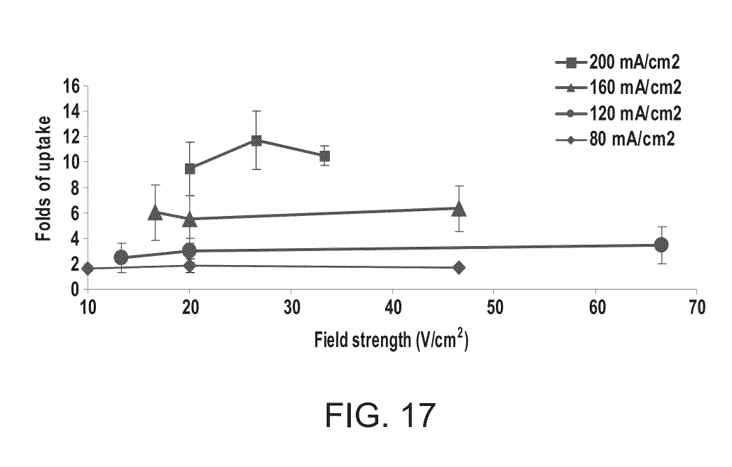

In order to determine the relative contributions of the electric field and the electrolytic reactions to the observed electric enhanced uptake, a three compartment exposure cell (FIG. 15) was constructed, by inserting two highly porous tortuous membranes. These membranes possess very low electric resistance when placed in physiological solutions. Therefore one should expect to have equal unattenuated electric field in each of the three compartments as compared with the same exposure chamber, in the absence of the two porous membranes. This was verified by demonstrating unaltered electric current in the presence and the absence of the two membranes. At the same time the membranes retard the diffusional or electrophoretic transport of the electrolytic products between the compartments. This was verified by the fact that no significant pH change was observed in the central compartment following one minute of exposure to the EPT while both acidification and alkalanization were apparent in the anodic and the cathodic compartments respectively. A similar situation was observed with two low molecular fluorescent dyes (lucifer yellow and tryphan blue), where no detectable penetration of these probes either from the anodic or from the cathodic compartment into the central one during the one minute of exposure. The results obtained show clearly that the uptake is enhanced only in the anodic compartment, while no changes were observed either in the central compartment or in the cathodic one (FIG. 16). These findings suggest that the electric enhanced uptake is not driven by the electric field but is associated with electrolytic products formed in the anodic compartment. This conclusion is supported by data shown in FIG. 17, demonstrating that the enhanced uptake depends on the intensity of electric current density rather than on the electric field strength. In an exemplary embodiment of the invention, when treating cells, such cells are provided in an anodic compartment of a two (anodic/cathodic) or three part cell. Such device can be built to be implanted in the human body and/or constructed inside the body, by positioning of membranes and electrodes. Optionally, the position of the electrodes relative to tissue to be treated is selected to achieve a desired pH at the tissue. Optionally or alternatively, a buffer solution is provided at the tissue to be treated to control pH. Optionally or alternatively, for example, when mainly electrical effects are desired, the anode is surrounded by a void, for example, caused by pushing away tissue, to avoid or reduce pH effects on viable tissue and/or tissue not to be treated. In an exemplary embodiment of the invention, the anode is provided inside a porous balloon (e.g., one which can be inflated using fluid but allows ion movement across its wall) or expandable cage structure or other covering.

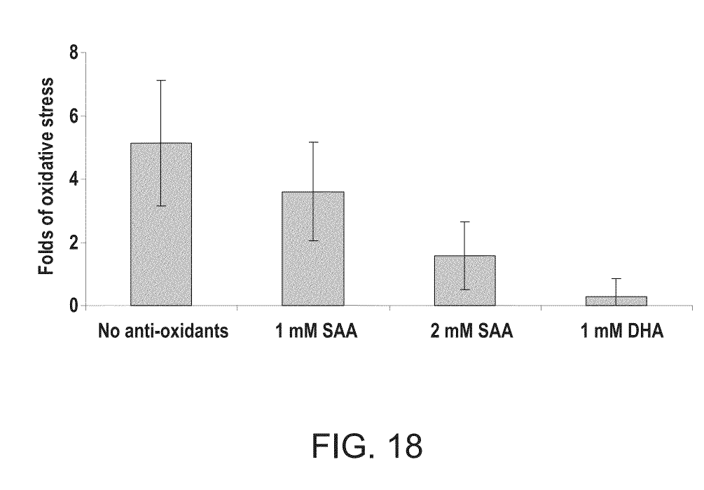

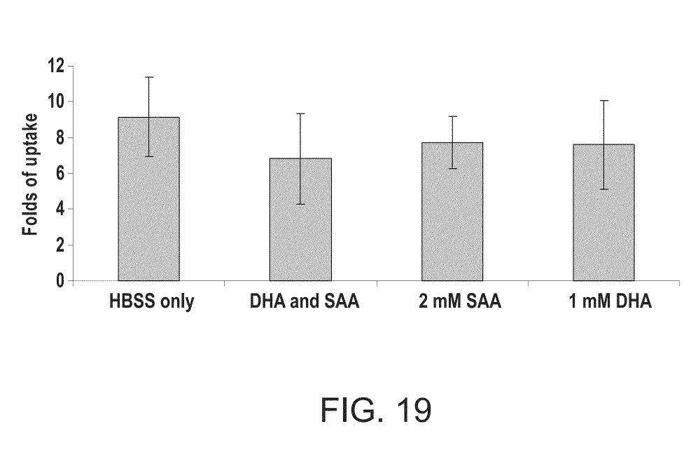

Electrolysis at the anode's face produces radical oxidative species as well as increases hydrogen ion concentration. Oxidative radicals are short lived yet their effect on the living system can be both pronounced and prolonged. The existence of oxidative intermediates in the extracellular medium following its exposure to EPT (Electric Pulse Train) or other electric field, is demonstrated. Their oxidative effect can be quenched by reacting with 2 mM SAA in the extracelluar medium during exposure to EPT. In FIG. 18 is its shown that intracellular OS (oxygen stress) developed in the cells present in the anodic compartment and that such OS can be substantially reduced using extracellular ascorbic acid (SAA) or entirely abolished using intracellular ascorbic acid (DHA). However, in the presence of both 2 mM SAA and 1 mM DHA, only a minor decrease could be found in the EPT induced uptake dextran-FITC compared to the constitutive level of uptake (FIG. 19). These findings indicate that even though elevated levels of OS are found in the cells during their exposure to EPT, it has only a minor, insignificant, impact on the phenomena of electric induced uptake.

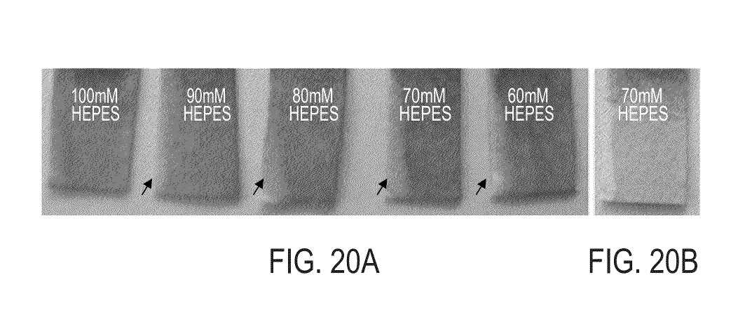

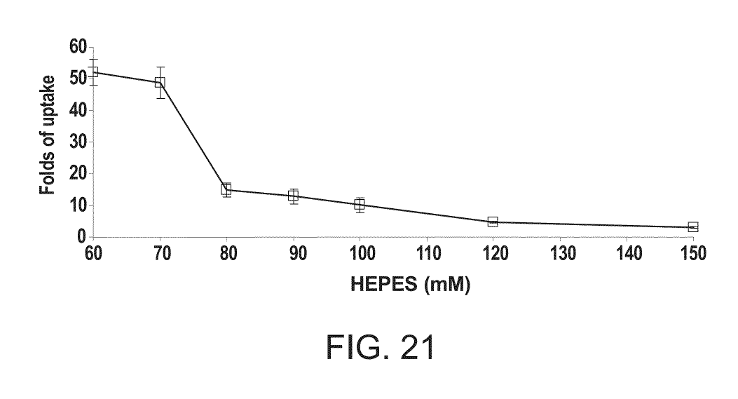

Anodic hydrolysis produces excess of hydrogen ions, and hence leads to a marked decrease in pH values. Due to the high buffer capacity of the external solution (100 mM HEPES), the anodic acidity induced is of transient nature and its effect diminishes both temporally and spatially (FIG. 20). As shown, cells away from the electrode face are less susceptible to the acidic effects. As the solution buffering capacity is lowered, the area of low pH widens, engulfing more cells and consequently affecting them, leading to higher uptake levels as evident from FIG. 21. In an exemplary embodiment of the invention, a range of effect of an electrical therapy (e.g., not necessarily PIU) is modified by changing a buffering environment of the cells being treated, in addition to or instead of modifying an electric filed application parameter. The electrolytically produced low pH environment is shown to be capable of being mimicked by adding hydrochloric acid to the cell's suspension. It is observed that the extent of dextran-FITC uptake is affected by the solution pH with an IC₅₀ in the range of pH=3.5. As shown in FIGS. 1A-1C, pH induced uptake is much lower at pH 5.8, yet still significant relative to the cell's constitutive uptake at pH 7 (FIG. 1A, inset).

The viability of cells exposed to similar pH changes has been examined. The short one minute exposure to extremely low pH (ELpH) had small effect on cell viability and suggests that fast uptake can be achieved for short times (e.g., several minutes, such as 20 minutes or less or 5 minutes or less or 1 minute or less), at least. Only 10% of the cells were stained positive by PI, used as an indication of a compromised integrity of the plasma membrane of these cells and their necrotic state. No increase in annexin labeling was found two hours following ELpH exposure, and no decrease in cell number following 24 hours of cell cultivation, relative to an unexposed cell culture was found either. All this indicates that cell viability is only marginally affected by the short exposure to ELpH.

The finding shown in FIG. 21 and FIGS. 1A-1C support the conclusion that the elevated concentration of hydrogen ions due to EPT is the major and most prominent contributor to the electric induced uptake.

The possibility that ELpH induces an increase in membrane permeability can probably be ruled out since exposing the cells to ELpH in the presence of PI was not accompanied by a rapid increase in nuclei fluorescence as normally happens when PI penetrates the plasma membrane and binds to nucleic acids.

The following tables show a connection between pH and current. In these experiments, cells were exposed, for one minute, in the presence of 10 μM Dextran-FITC, to chemicals and/or electrical fields. The values in the tables are shown as folds relative to the uptake in cells unexposed to any of the treatments. As can be seen, Exposing the cells to 200 mA in 100 mM HEPES buffer is substantially the same as exposing them to ˜pH 3.5. The results are expected to be substantially independent of cell type and molecule used, except for a cell-specific change and a molecule specific change. The first table shows uptake folds and standard deviation when the exposure was to a chemically induced pH change

Folds Uptake

| pH | mean | SD |
|---|---|---|
| 1.6 | 48.09023 | 7.699552 |
| 1.9 | 42.47456 | 2.630711 |
| 2.5 | 47.61606 | 8.806872 |
| 2.9 | 45.54382 | 4.447135 |
| 3.1 | 41.25006 | 4.477876 |
| 3.3 | 20.14338 | 1.15015 |
| 3.5 | 9.574667 | 1.612258 |
| 3.7 | 7.678626 | 1.976118 |
| 4 | 4.775733 | 0.524769 |
| 4.7 | 3.85572 | 1.168029 |
| 5 | 2.594479 | 0.916997 |
| 5.8 | 2.636394 | 0.401966 |
| 6 | 1.482987 | 0.457736 |
| 6.25 | 1.61866 | 0.78179 |
| 7.4 | 1 | 0.061621 |

A second table shows that when applying an electric field having a density of 200 mA per square cm, (at 100 mM HEPES), the same results are achieved

Folds Uptake

| V/cm | mA/Cm\hat{ }2 | Fold | SD |
|---|---|---|---|
| 20 | | 9.134157 | 2.217004 |
| 26.6 | 200 | 11.73415 | 2.300328 |
| 33.3 | | 10.48628 | 0.759543 |
| 16.6 | | 6.016938 | 2.164264 |
| 20 | 160 | 5.476251 | 1.893391 |
| 46.6 | | 6.34 | 1.8 |
| 13.3 | | 2.448618 | 1.153828 |
| 20 | 120 | 3.014594 | 0.957136 |
| 66.6 | | 3.447481 | 1.449856 |
| 10 | | 1.619816 | 0.218777 |
| 20 | 80 | 1.824273 | 0.512907 |
| 46.6 | | 1.694299 | 0.10814 |

A third table shows that modifying the amount of Hepes can change the effect of an electrical field:

Folds Uptake

| mM HEPES | mean | SD |
|---|---|---|
| 60 | 51.97857 | 4.091665 |
| 70 | 48.84179 | 4.954718 |
| 80 | 14.85769 | 2.32419 |
| 90 | 12.80557 | 2.389428 |
| 100 | 10.05917 | 2.259907 |
| 150 | 3.106448 | 0.315623 |
| 120 | 4.613101 | 0.296984 |

In an exemplary embodiment of the invention, tissue is target by a current by selecting tissue to be targeted that has a lower buffering capacity or lower pH and/or by artificially lowering pH or washing away a buffer. In a particular example, in blood vessels, slow blood flow (or replacing blood with a non-buffering fluid) can assist in targeting endothelial cells, while high blood flow can protect endothelial cells by washing away any pH effect of the field. In one example, cells near an anode are protected (e.g., reduced uptake) by locally applying a buffering solution or a high pH solution and/or continuous washing. Any of these may be provided, for example, by the electrode itself, by a tube, for example, which surrounds the electrode or by using a porous conduit as an electrode. Optionally or alternatively, such a tube is used to provide a pulse of material to be taken up and/or modify membrane characteristics. Optionally, a first material provided is used to protect a cell form an effect of a second material or enhance its effect.

Exemplary Implementation

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

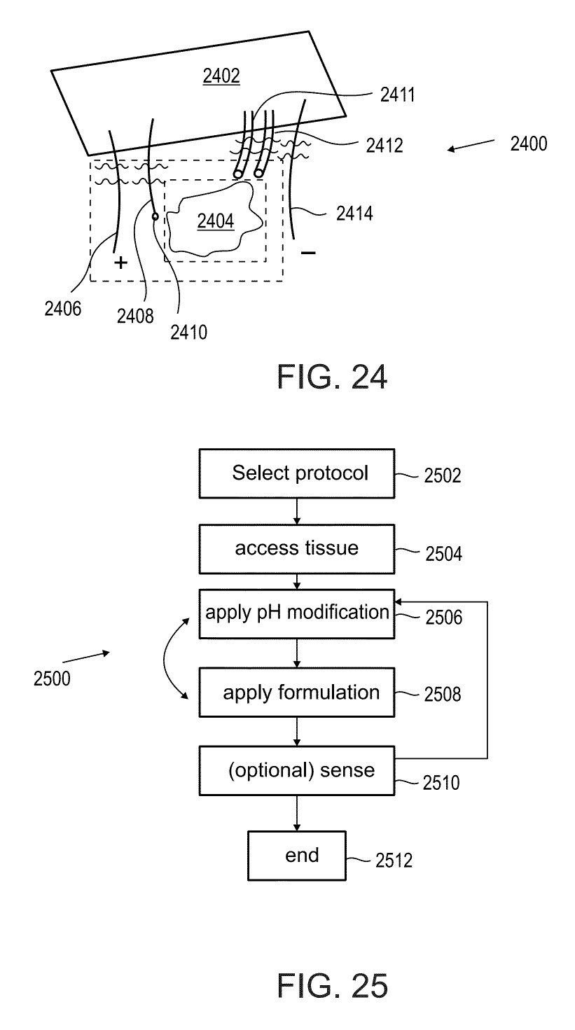

Referring now to the drawings, FIG. 24 illustrates an exemplary system 2400 for treating cells and FIG. 25 illustrates an exemplary method 2500 of treating cells, in accordance with an exemplary embodiment of the invention.

FIG. 24 is a schematic illustration of a system 2400 including a controller 2402, including, for example, electricity generating circuitry and logic circuitry, which electrifies an optional anode 2406 and a cathode 2414, so as to treat tissue 2404, in accordance with an exemplary embodiment of the invention.

In an exemplary embodiment of the invention, the voltage and current density are sufficient for electrolysis e.g. for a plain platinum electrode the threshold is 1.2V at current densities approaching zero or 0.2V at current densities of 10 mA/cm². Optionally, the electrode is selected to be a high charge electrode suitable for delivering a high charge. Optionally or alternatively, the electrode does not cause local concentrations of electric field. Alternatively, such concentrations are desired, for enhancing electrolysis, for example.

Exemplary application durations (net of interpulse durations) include, 30 seconds, 1 minute, 3 minutes, 5 minutes, 10 minutes, 15 minutes, 30 minutes, 1 hour and 3 hours, or smaller or intermediate or larger durations. Exemplary formulation concentrations are 1 ppb, 100 ppb, 1 ppm, 100 ppm, 1000 ppm, 2000 ppm, or smaller or intermediate or larger concentrations. The concentrations may be selected, for example, to avoid cell damage for the treated and/or untreated cells and/or to determine, e.g., together with pH, treatment time.

Optionally, a sensor probe 2408, with a sensor 2410 is provided, for example, for sensing pH or a concentration of a formulation to be taken up by tissue 2404. Controller 2402 optionally controls the electric field in accordance with sensing results and/or a program of uptake enhancement.

Optionally, a source of formulation 2411 is provided. Optionally, a pump (not shown) is provided with controller 2402 to pump the formulation as needed. Optionally, source 2411 includes a needle which is penetrated at or near tissue 2404 or into a vascular bed thereof. Optionally or alternatively, a source of acidifying material (or other hydrogen ion source) 2412 is provided, instead of or in addition to the electrodes. Optionally, the formulation and acidifying material are mixed and provided together.

In an exemplary embodiment of the invention, anode 2406, optional sensor 2410 and/or sources 2411 and 2412 are provided in a housing (shown as a dotted line), for example, a catheter or endoscope or needle. Optionally, cathode 2414 is provided at a remote location, optionally outside the body. In an alternative embodiment, protons are provided by an entity, for example, a polymer or cage which spontaneously degrades and releases hydrogen ions. Optionally, control is provided by selectively isolating such an entity from the body tissues.

FIG. 25 is a flowchart of a method 2500 of using system 2500, in accordance with an exemplary embodiment of the invention.

At 2502, a treatment protocol is selected, including, for example, one or more of duration, pH, formulation(s)—several can be applied, for example, in series or simultaneously, number of pulses, interpulse durations. Optionally, two (or more) tissue areas with overlap are treated, such that the overlap tissue receives two different treatments.

At 2504 the tissue is accessed, for example, by a needle or catheter or into an open wound, or by removing the tissue from the body.

At 2506, the local pH is modified, for example, using a locally applied anodic current or by other means described herein of hydrogen ion provision.

At 2508, optionally simultaneously with or before 2506, a treatment formulation to be taken up by the tissue, is provided.

Acts 2506 and/or 2508 may continue for a set time and/or in accordance with a more complex protocol, such as pulsed delivery of pH reduction. In case of a high buffering capacity in the specific location in the body where the enhanced uptake is intended to take place, prewashing with a low buffer solution prior to the formation of a low pH at the same site, is optionally performed.

At 2510, the formulation concentration, pH and/or tissue physiological reaction are optionally determined. Depending on the selected protocol, the pH modification and/or formulation may be, for example, adjusted, continued and/or stopped.

At 2512, the process is completed. If system 2400 is implanted, the implanted device may be removed or optionally left in for later use. In some embodiments, the delivery system is a material-eluting matrix, which may be formulated and selected to have a desired effect and left in the body, with the matrix optionally bio-dissipating after a time.

Blood cells can be subjected to acidic treatment and consequently uptake in an ex-vivo device, before they are returned to the patient. In an example for such device, blood cells are isolated and flown through a porous hollow fiber with a cutoff appropriate for the drug or particle to be introduced into these cells. The hollow fiber path transverses one chamber which contain the designated drug or nanoparticles in an acidic solution in the range of, for example, pH 4 to pH 6 and then another chamber containing a physiological neutral solution. Alternatively, pH is controlled using an anodic current. Finally the cells are collected and administered back to the patient from whom they were taken.

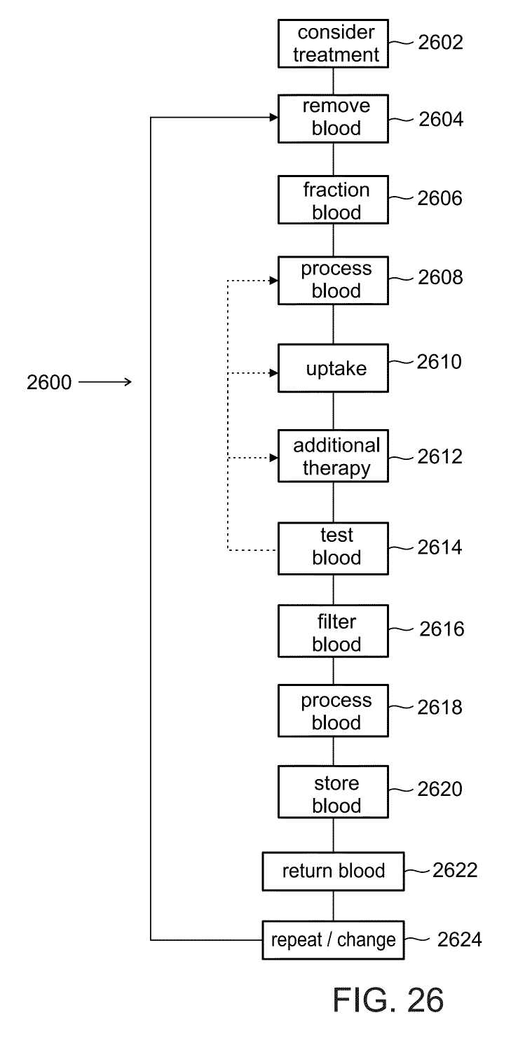

FIG. 26 is a flowchart of a method 2600 of treating blood cells, in accordance with an exemplary embodiment of the invention.

At 2602, a treatment to be applied is considered.

At 2604, blood is removed from the body. In some embodiments, the blood processing is performed using an implant, for example a hollow tube design such as described with reference to FIG. 29, which acts as a shunt for arterial and/or venous flow.

At 2606, the blood is optionally fractioned, so only some of the blood is actually treated.

At 2608 (optionally before 2606), the blood is optionally processed, for example, cleaned or sterilized or some proteins added or removed, for example, using anti-bodies or filters.

At 2610, the blood is exposed to materials to be uptaken in accordance with the methods described herein

At 2612, additional therapy is optionally applied to the blood.

At 2614, the blood is optionally tested for a desired effect of the treatment, for example, assayed to determine percentage of affected cells. Treatment and/or processing may be repeated and/or modified based on the results.

At 2616, the blood is optionally filtered to remove dead and/or damaged cells.

At 2618, the blood is optionally washed and/or otherwise processed, and/or materials added, to make it suitable for physiological use.

At 2620, the blood is optionally stored, for example, in cooled, frozen or dehydrated form. In an alternative embodiment, the blood is continuously removed, processed and returned to the body.

At 2622, the blood is injected into a body, optionally of the original subject form which it was removed.

At 2624, depending on the effects of therapy, the process may be repeated or changed.

It should be noted that this method may also be applied to cells other than blood, which may be removed form and returned to a body, for example, tissue plugs or stem cells. Optionally, such tissue is broken down into a suspension of cells at act 2608.

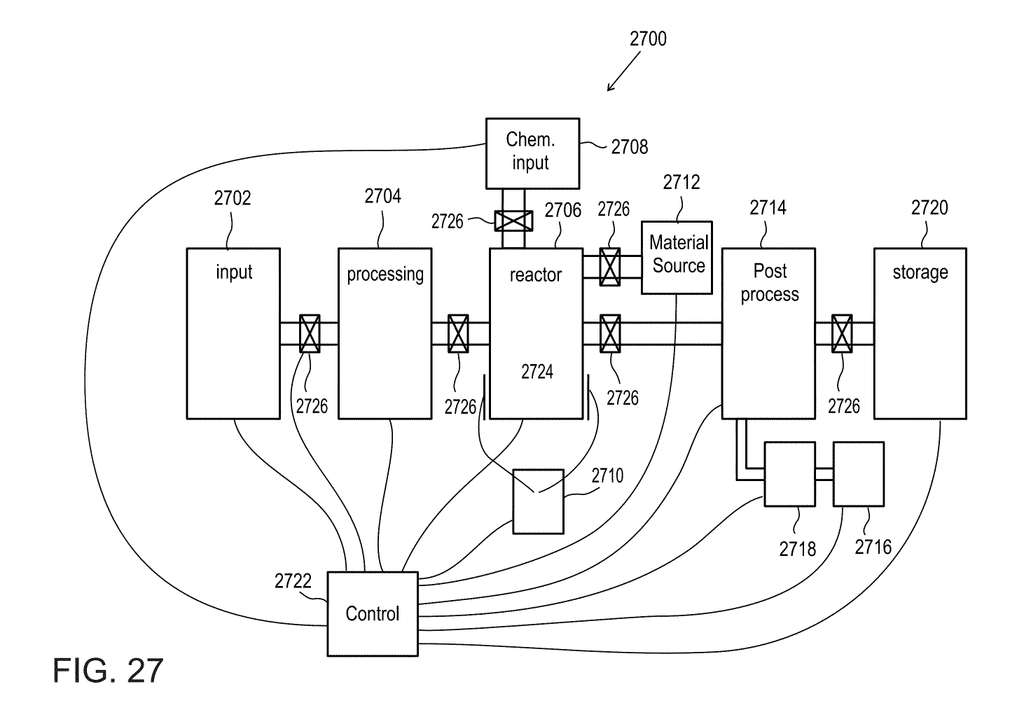

FIG. 27 is a schematic block diagram of a stream processing system 2700, in accordance with some embodiments of the invention, which may be used, for example, for treating blood and/or cell cultures.

An input 2702 provides a fluid containing cells to be treated. The cells enter an optional processing stage 2704, which may, for example, reduce the amount of fluid, add solution or filter the fluid. A reactor 2706 has, for example, one or both of chemical input 2708 for adding acid material and/or buffer solution and one or more electrical field applicators 2710. Optionally, the conductors are designed to cause a known and relatively uniform current within the entire cross-section of reactor 2706 and/or a known part thereof. Optionally or alternatively, the contents of reactor 2706 are agitated so that the cells can enter and leave such treatment area.

In an exemplary embodiment of the invention, a material to be taken up is provided by an inlet 2712, into the reactor.

Multiple reactors may be chained, for example, if multiple processing stages are provided or if multiple treatments are provided.

At output from the reactor, a post-processing stage 2714, for example, a filter or washing station processes the cells. Optionally, some cells are removed for manual or automatic quality control for example, using an imaging system 2716, which identifies a percentage of treated cells and/or a percentage of dead cells. Optionally, a cell manipulation unit 2718 is used to pre-process such imaged cells, for example, by adding a stain thereto. For example Annexin may be used for apoptosis, Propidium Iodide for membrane integrity and/or MTX for mitochondrial activity.

Finally, a storage unit 2720 is optionally provided to store the cells and/or send then back to a source location.

In an exemplary embodiment of the invention, a controller 2722 controls the process, optionally using a pH or other sensor 2724 in the reactor and optionally controlling the flow of materials by controlling one or more valves and/or pumps 2726 on the material sources. In some cases, the process is carried out without a controller, for example, by allowing blood to flow through pre-setup channels with acidifiers and materials to be taken up precalculated to have a desired uptake effect.

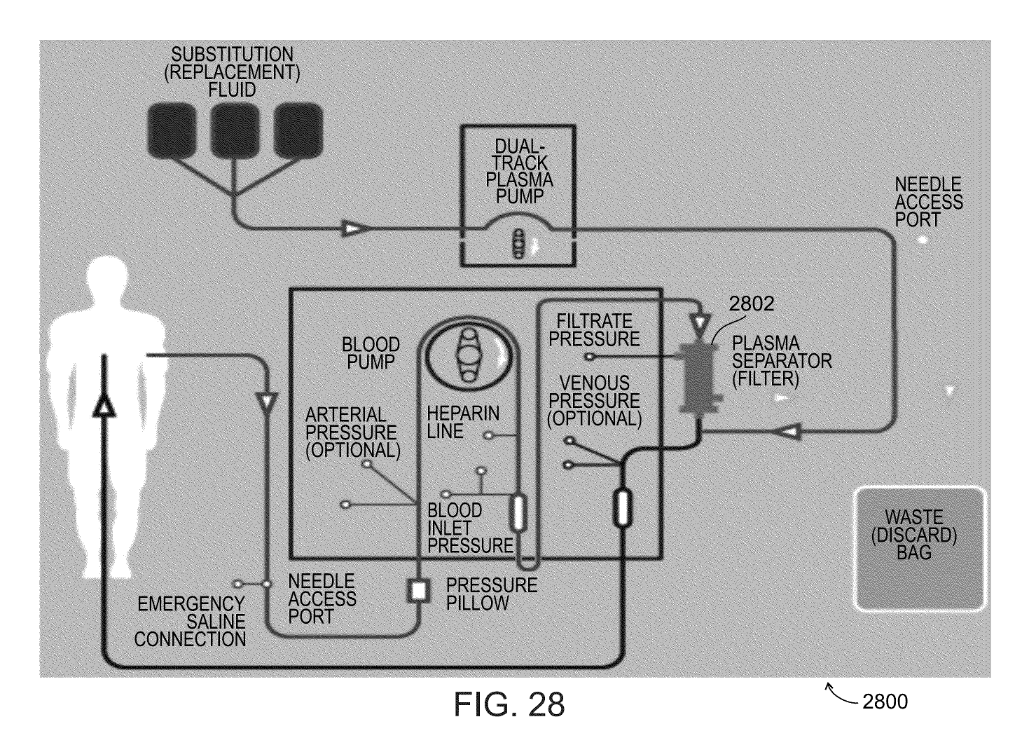

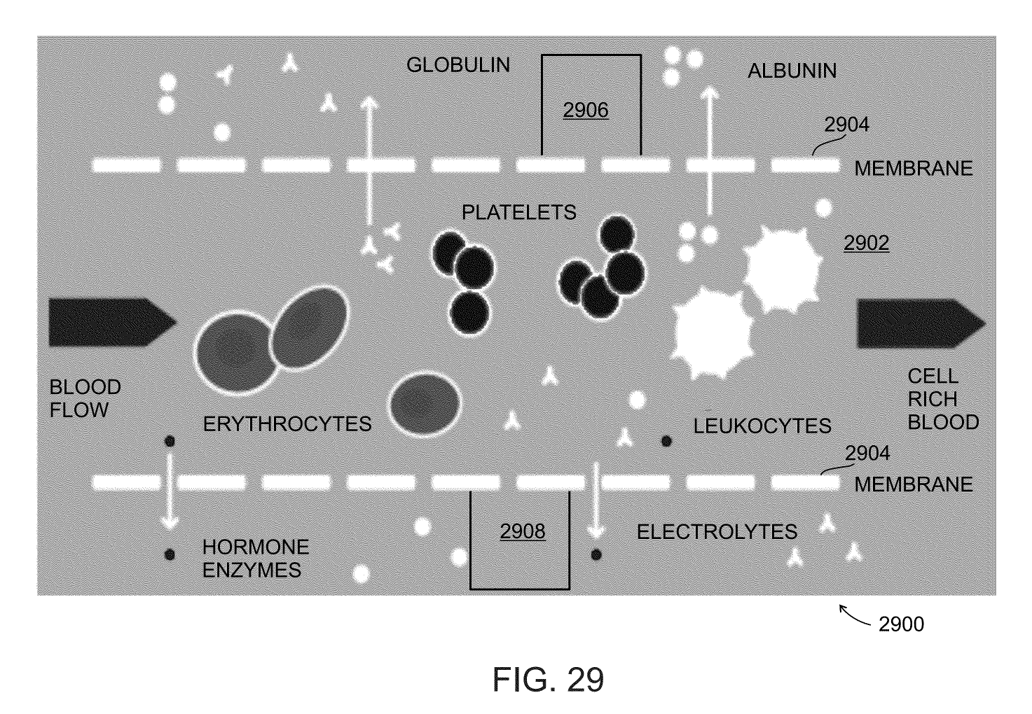

FIG. 28 is a schematic showing of an aphoresis system 2800 (e.g., of a type known in the art) for removing blood from a body, modified in accordance with a new exemplary embodiment of the invention. As shown, blood is removed from a body, optionally pumped and separated. Optionally, a blood modifying element 2802 is provided instead of the shown plasma separator or in series with it.

FIG. 29 is a schematic showing of a hollow-fiber reactor 2900, useful in accordance with some embodiments of the invention. A flow of blood (or other suspended cells) 2902 passes adjacent to or enclosed by one or more porous membranes 2904. As shown, various proteins and/or ions optionally pass through the membrane. In some embodiments, fluid outside the membranes is matched with the blood, to prevent such migration. In an exemplary embodiment of the invention, one or more chambers outside of the membranes are used for uptake control. In an alternative embodiment, the membranes themselves include materials for uptake control. Optionally or alternatively, one or more electric field generators is used to apply a field across and/or on the flow.

In the example shown, a first chamber 2906 is open to the flow and releases (e.g., through the membrane), for example, a material to be taken up. Optionally or alternatively, a second chamber 2908 is open to the flow and releases (e.g., through the membrane) acidification materials. Optionally, such materials are provided after buffering ions are removed from the blood and/or in an amount sufficient to overcome such buffering. Optionally, buffering ions and/or other electrolytes are added back to the blood, as needed, after uptake is complete. Optionally or alternatively, a pH-balancing fluid is added to stop the uptake.

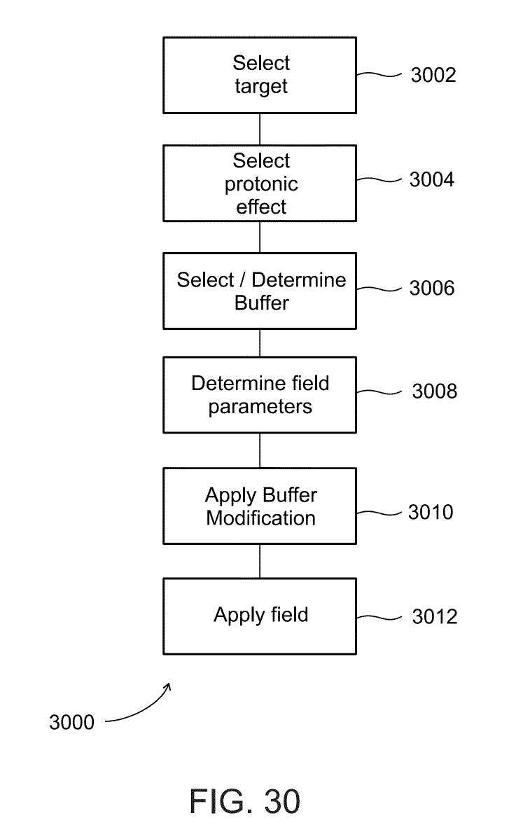

FIG. 30 is a flowchart 3000 of a method of electrical field application, in accordance with an exemplary embodiment of the invention.

At 3002, a target tissue is selected. Optionally, the selection is based on a determination that a tissue is more sensitive to electric fields due to a reduced buffering capacity or low pH. This may be true of some cancerous tissue.

At 3004, a desired proton-mediated effect is selected, for example, uptake. This may include selecting a material to be uptaken and/or an uptake assisting or blocking material.

At 3006, a buffering material and/or local pH is selected or determined to exist.

At 3008, the field parameter (e.g., one or more of current, repetition rate, interpulse delay, pulse length, number of pulses in a train, voltage) to be applied are determined to match the desired target effect and buffering effect. Acts 3004, 3006 and 3008 maybe changed in order and/or performed together, optionally by a calculation circuitry.

At 3010 the buffer modification, pH modification and/or material to be taken up are applied.

At 3012, the field is applied.

In exemplary embodiments of the invention, the various devices for treating cells are programmed to operate in accordance with parameters described herein, optionally taking into account cell specific properties such as sensitivity to pH.

### Some Exemplary Embodiments for Selective PIU

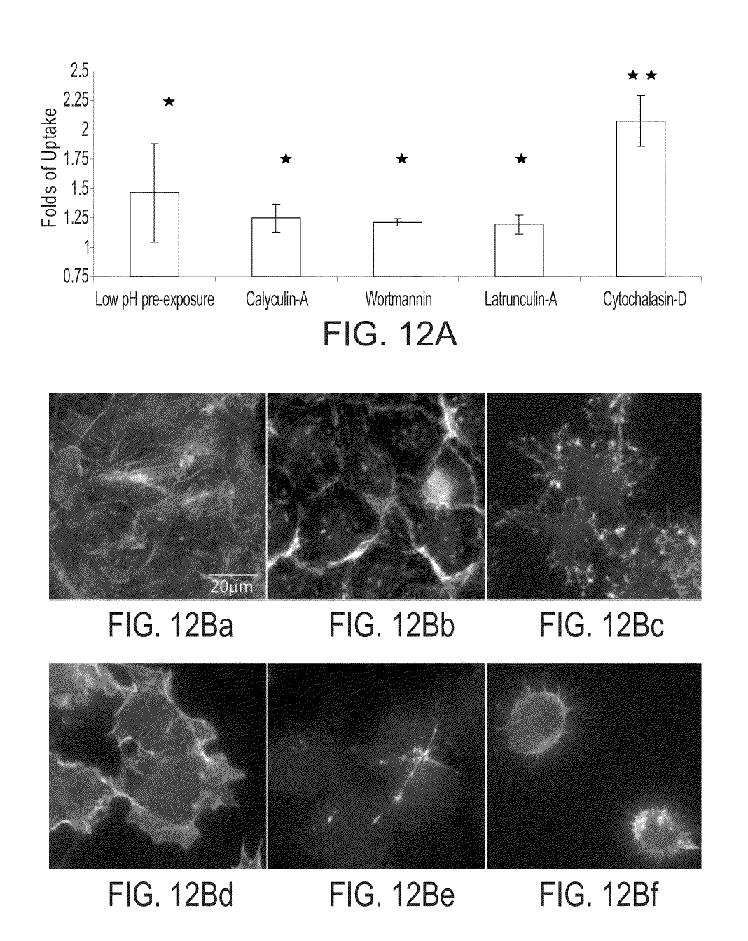

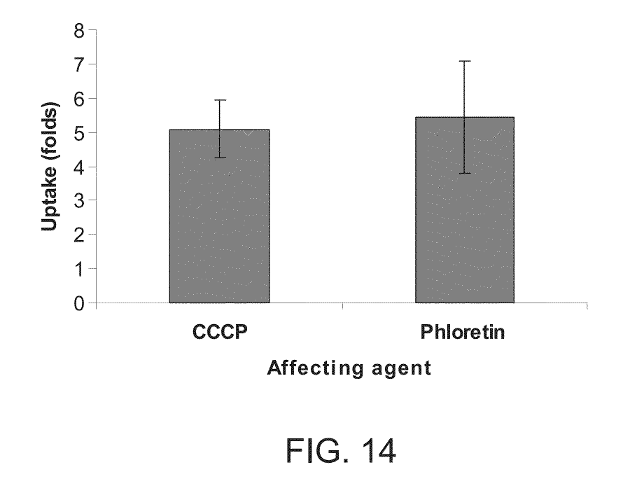

The PIU can be augmented or attenuated in certain cell populations, based on some environmental or physiological characteristics. For example, one or more of the following may be applied:
1. The extent of uptake is linearly dependent on the tissue temperature (FIG. 5). For example, a tissue can be targeted for enhanced uptake according to its temperature difference from the surrounding tissues. Such difference can be enhanced by actively cooling some tissue sections and not others.
2. Cells in confluent tissues where cells are packed in high density or posses high level of cell to cell connectivity, undergo a lesser level of uptake then non confluent, loose cell populations (FIG. 7). Example for cell tissue which may present higher levels of uptake are:
   a. Tissues recovering from trauma, injury, or surgery
   b. Tissues with un-organized structure
   c. Tissues with diffused cell population
   d. Tissues with high level of perfusion
   e. Expending tissues with rapid proliferation
   f. Migrating cells with are not anchored into the tissue
   g. Suspended and unattached cells
3. Disruption of the cells' actin cytoskeleton can increase the rate of PIU, in particular when the actin fibers are severed and induced to branch (FIG. 12). Similar disruption of the cytoskeleton exists in dividing cells and migrating cells. Chemical treatment or selective targeting of dividing cells may thus be provided. The electric potential across the plasma membrane affects the cell ability to perform PIU (FIG. 13):
   a. Hyper-polarization of the cell membrane is shown to reduce the rate of PIU in non-excitable cells. This can be provided, for example, by changing the ionic composition of the bulk solution.
   b. De-polarization of the cell membrane is shown to increase the rate of PIU in non-excitable cells. Membrane depolarization is reported to be found cells bordering wounded tissues.
4. Reducing the dipole potential of the plasma membrane (intra membrane potential) has the ability to increase the rate of PIU (FIG. 14). Such effects can be reproduced by other agents that affect the plasma membrane dipole, for examples anesthetics, where local anesthetic applied to an organ is used to allow that organ to uptake at a higher rate.
5. In an exemplary embodiment of the invention, one or more treatment parameters are selected to increase or maximize the difference in uptake between different cells in a target tissue region.

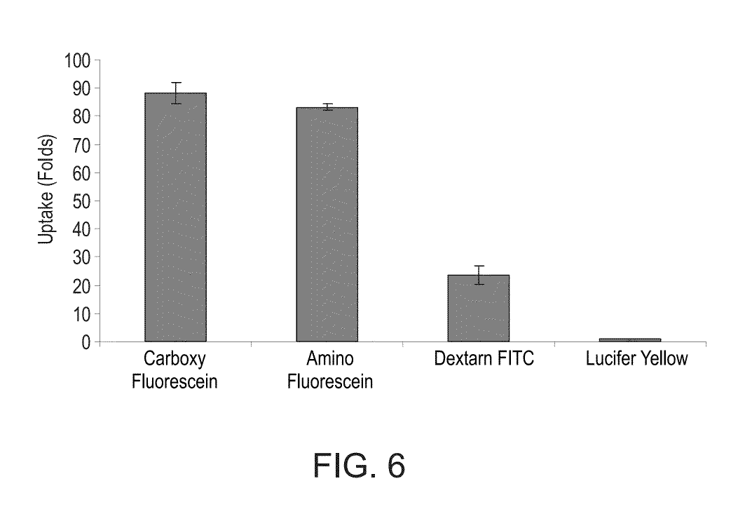

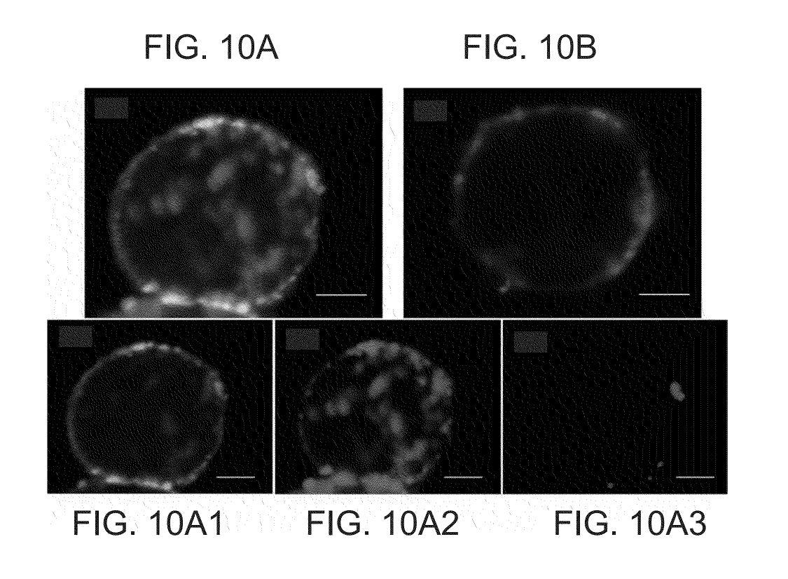

The level of PIU can be controlled according to the nature of the molecules or particles that are intended for delivery:
1. The rate of PIU in the cells is linearly correlated to the concentration of molecules outside the cells (FIG. 8).
2. Particles with a charged surface undergo better uptake than non-charged particles (FIG. 10).
3. The absorbance of molecules to the cell surface increases the rate of their uptake (FIG. 6):
   a. Molecules with some hydrophobic areas or a single electric charge are better delivered than molecules with several charges, possibly as they are less replused.
   b. Charged polymers (such as polysaccharides) are better delivered than small charged molecules, possibly as they better attach to membranes.

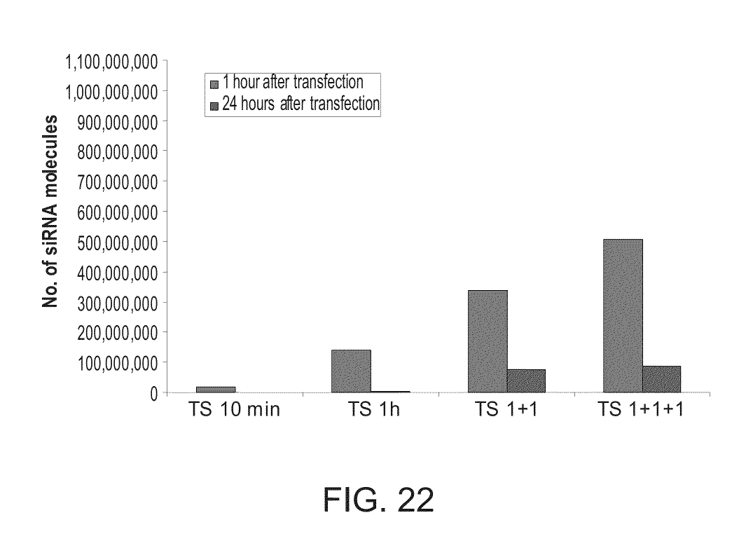

The exposure of cells to a regime of repeated treatments can be used for increasing the number of molecules that are uptaken by the cells, without inflicting irreversible damage. In such treatment regime, uptake is repeatedly induced by a train of cell exposures to low pH (for example pH 5) separated by exposures (e.g., long enough to stabilize internal pH) to physiological pH (e.g. pH 7.4), or higher (e.g., to provide an overshoot effect). For example, increase in the number of siRNA found inside cells is demonstrated following repeated exposures to low pH solution (FIG. 22).

Example for Cancer Therapy

Targeting may be provided. For instance nano-size magnetic beads coated with thermosensitive polymers (e.g. pluronic gel), which are associated with a chemotherapeutic drug are provided. These nanoparticles will accumulate in the cancer site by an enhanced permeability retention (EPR) mechanism. Other targeting methods may be used as well.

The release is optionally initiated by temperature elevation (via exposing the tumor area where the magnetic beads concentrate to external AC electric field) which will lead to temperature rise of the magnetic beads and consequently will release the chemotherapeutic drug and hydrogen ions (e.g. Eudragit L100-55). In an alternative embodiment, ultrasonic heating or liposome decomposition is provided.

Alternatively or additionally to releasing hydrogen ions, using the uptake enhancement methods described herein may be used to enhance uptake.

For example, uptake of non-permeable chemotherapies such as Taxol can be enhanced. Optionally or alternatively, such release optionally together with uptake enhancement (e.g., using an electric field) may be used for assisting in drug penetration into other cells which are resistant, for example, into brain cells, across the blood-brain barrier. Other methods of causing pH increase may be used as well.

Examples of Applications for Blood Cells
1. Anti Infection therapy is based on inducing uptake of drugs in macrophages. One example is the uptake of anti HIV drugs such as AZT and DDI (nucleoside analogues) which serve as reverse transcription inhibitors and hamper the virus proliferation. Additional diseases which may be treated are Leishmania and Listeria, wherein anti-biotics are inserted into the cells.
2. Red blood cell (RBC) drug carriers are erythrocytes uploaded with drugs formulations stabilized as polymers or nanoparticles. These formulations are then gradually released from the cell to the blood for example by the action of endogenic enzymes. One example is the maintenance of a constant blood level of corticoids anti inflammatory drugs. Additional examples are for release of one or more of Erythropoietin, growth hormone, testosterone and/or antibiotics.
3. RBCs may be used as a therapeutic vehicle by loading them with drugs and injecting them to a local site, for example a wound, a tumor or a distressed organ.
4. Improving the oxygen capacity of erythrocyte allows it to carry more oxygen to the body tissues and more CO₂ away from it. One example is erythrocyte uptake of perfluorocarbon nanoparticles which posses 20 time greater oxygen capacity than hemoglobin. Such treatment may be applied, for example, to a patient with reduced amount of blood and/or lung capacity, with the shell of the red blood cells possibly preventing adverse effects.
5. Refurnishing the anti-oxidative competence of erythrocytes by inducing the uptake of anti-oxidants. Example formulations are glutathione, ascorbate or tocoferol. See, for example, M. Firosari at al 2007, Asian Journal of Biochemistry 2(6) p 437 “Activities of anti-oxidative Enzymes, Catalase and Glutathione Reductase in Red Blood Cells of Patients with Coronary Artery Disease”.
6. RBCs can be transformed into circulating bioreactors by loading them with enzymes that are capable to modify (e.g. degrade, catalyze) various substrates found in the blood circulation. For example cells loaded with the enzymes catalase and/or superoxidedismutase will have an improved capacity to decompose super oxides and hydrogen peroxide from the blood flow.
7. RBCs can be loaded with therapeutic molecules that are aimed for the liver or spleen. When aged, RBCs are targeted to these organs for degradation and their content is released in a timely manner.
8. RBCs loaded with large molecules, for example, >=70 kD. Such molecules can be, for example, proteins, enzymes, nanoparticles, poly saccarides and/or other molecules which do not naturally traverse the membrane of the RBC. Such molecules can also be loaded in to other cells types, for example, as described herein.
9. RBCs loaded with multiple materials, by sequential loading of each material using methods described herein.
10. In the experiments described below with reference to RBCs there was no hemolysis visible after uptake (though handling can cause some RBC death). This is very different from methods known in the art and makes it practical to use RBCs harvested from and returned to a patient.

General

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non limiting fashion.

### Examples I

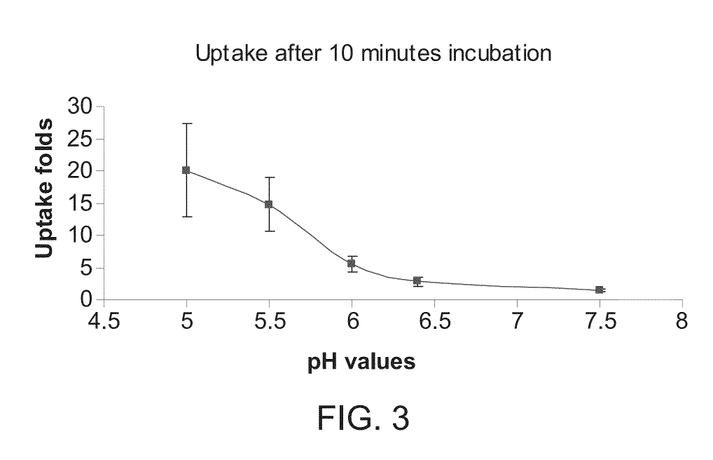

### Relating to FIGS. 1-4

Methods in General

Cell Harvesting

Caco-2 cells were cultivated, and were harvested when reaching 80% confluence by the use of trypsin-EDTA. The cell suspensions were immediately diluted in full medium (DMEM with 4.5 mg/ml glucose, 10% FCS), centrifuged and re-suspended in recovery medium (HBSS or PBS).

Uptake of Macromolecules

Cells in suspension are exposed to acidified solution in the presence of the fluorescent macromolecular probe (70 kDa dextran-FITC, 10 μM) for the period of time required by experimental setup. Once exposure is terminated the suspension is transferred to a small vial where it is either incubated at 37° C. or immediately cooled down to 4° C. All samples are collected together on ice and then simultaneously analyzed.

Acidification is imposed by supplementing the suspension with 10 mM MES buffer and the concentrations of HCl required for reaching the experimental pH level.

The cells are washed by being centrifuged, re-suspended with fresh DMEM (without phenol-red or FCS), incubated at room temperature with probe specific digestive enzymes, where appropriate, centrifuged and washed again.

For FACS analysis, Trypan-blue (0.01%) and PI (2 μg/ml) are added to the cells suspension to quench residual extracellular fluorescence and stain necrotic cells, respectively.

Alamar Blue Assay:

Alamar blue is an indicator that changes both absorption and fluorescence in response to chemical reduction of growth medium resulting from cell growth. For a viability test, cells were exposed to acidic conditions, buffered and washed. Fluorescence (485/595) was measured before and after incubation with alamar-blue (for 30 minutes period).

BCECF-AM Assay:

Membrane-permeate AM ester of pH sensitive carboxyfluorescein. Once cleaved by cytosol esterase the dye is efficiently retained in the cell. Loading the dye into cells was done by incubation with 1 μM BCECF-AM for 30 minutes, followed by double wash. Fluorescence was measured at in a ratio mode employing 440/485 nm excitation and 535 nm emission.

Electron Microscopy (EM):

Suspensions of cells were exposed to acidity in the presence of 10 nm gold particles for 10 minutes, followed by dilution, buffering and double wash with HBSS. The suspensions were incubated at RT for 30 minutes, followed by centrifuge and re-suspension in 10% glutar-aldehyde solution and immediate sedimentation. The cell pellet was treated by standard protocol of osmium staining, dehydration and epoxy blocking, followed by 5 μm thick slicing.

Results

The generality of the PIU phenomenon was validated by demonstrating its existence in five different cell lines. PIU of dextran-FITC as a function of the external pH, portrays a sigmoid-like relationship, with the steepest rise observed in the range of 3<pHₑₓₜ<4, for three different cell lines (FIGS. 1A-1C). These findings imply that the cells ability to respond to PIU is not a cell-line specific property but is a phenomenon common to many cell types. The relative high extent of PIU in TK6 cells could be attributed to the higher exposure of their cell surface to the macromolecules as compared with adherent cells. Among the adherent cell lines, the higher extent of PIU in HaCaT cells can be ascribed to their lower level of both constitutive endocytosis and membrane efflux pumps (MDR) activities (19) relative to Caco2/TC7 and COS 5-7 cell lines (20). The attenuation in PIU found with HT29 cells is most probably the product of their secreted mucus protective layer (21). Mucus is optionally taken into account when planning uptake in accordance with some embodiments of the invention. Another important question concerns the viability of cells under the harsh conditions of lower pH. While the exposure of cells to external low pH produces a short term stress, only a small fraction of the cell population (e.g. 2% and 5% for HaCaT and Caco-2/TC7 cells, respectively) undergoes necrosis immediately following exposure to the range of 7.4>pHₑₓₜ>5. The major part of the cell population is unaffected, in terms of cell number, red/ox activity and proliferating capacity. Moreover, the acidification was not accompanied by initiation of apoptosis as reflected by the negative results of the annexin assay.

The results also show that the increase in cell's fluorescent intensity following the uptake of dextran-FITC is not the result of compromised membrane permeability, since it did not augment the free entry of the small dye propidium-iodide into the cytoplasm. This dye, which is membrane impermeable, becomes fluorescent only upon binding to DNA and RNA.

Further studies centered around the near-physiological pH values (7.5-5) show the persistence of the low-pH induced uptake (LpHU) phenomena.

The kinetics of dextran accumulation in the cells could take the form of saturation curve under increasing exposure durations, becoming pronounced as the external pH is lowered, gradually shifting from a linear correlation at pH values close to pH 7.4, to a logarithmic shape at pH 5 (FIG. 2A). This apparent difference are studied by applying inhibitors of cellular efflux ATP binding cassette (ABC) pumps using cyclosporine-A or verapamil, or when the entire cellular ATP pool is depleted. Under these conditions, the cellular accumulation of dextrans as function of time shows a linear relationship (FIG. 4A). Thus the intracellular concentration of dextran seems to represent the balance between its afflux and efflux. The efflux of dextran was studied by measuring the time dependent decrease of intracellular dextran concentration that follows the cells exposure to low external pH. The observed decline rate in cellular fluorescence features an exponential shape at 37° C. (FIG. 2B), suggesting the efflux rate to be a metabolically driven process since it is abolished at 4° C. The constant prevalence of PIU would beto be followed by reduction in the cell area/volume ratio, consequently increasing the membrane tension and bending rigidity. The finding that PIU rate is constant for at least one hour of exposure, suggest no reduction in plasma membrane area does occur. Therefore membrane vesicles or invaginations are probably short lived and efficiently fuse back to replenish the plasma membrane area.

Endocytosis relationship with temperature above 10° C. takes a biphasic form, with linear correlation to temperature starting above 20° C. (FIG. 5). The activation energy associated with endocytosis, estimated from the Ahrenius plot, confirms this biphasic nature and having lower values above the 20° C. deflection point (22, 23). The temperature kinetics of PIU differs from these reports, by the lower temperature of the biphasic deflection point, between 9° C. and 4° C. When cooling complex mixtures of lipids such as those found in biological membranes, some lipid species may enter the gel phase while others are still in a liquid crystalline state. Such “phase separations” phenomenon in which gel and fluid phase lipids coexist, modify the lateral organization of membrane components. For membrane proteins sensitive to the physical state of surrounding lipids, the liquid to gel phase transition is generally associated with large changes in activity evidenced by the deflection in their Arrhenius plots positioned around 20° C. The shift in the deflection point of the PIU activation energy, relative to that of endocytosis, would suggest that PIU is independent of protein activity, and affected by the dependence of membrane bending modulus on temperature. Optionally, cooling and/or heating are used to control local amount of uptake.

### FIGS. 6 to 15

Comparing low pH dextran uptake of HaCaT cells in suspension with HaCaT cells in adherent culture (FIG. 7A) reveals a difference in their kinetic response to external low pH. While suspended cells maintain a constant rate of PIU during 60 minutes exposure period to pH 5.25, the PIU rate of adherent cultures begins 2.5 folds lower, and gradually increases during the course of 30 minutes exposure. In addition, in suspended cells membrane depolarization does not seem to affect uptake, but in surface adherent cells, membrane depolarization does affect uptake. In an exemplary embodiment of the invention, local injection of, for example, potassium is used to affect membrane polarization and depolarization and/or cross membrane difference, and thereby affect uptake.

Microscopic observation of the actin cytoskeleton reveals that during 30 minutes of exposure to acidic medium, a relatively slow reorganization of cytoskeletal elements take place in which stress fibers gradually disappear from cytoskeleton structure and are replaced by short twisted peripheral filaments (FIG. 12). Possibly this actin reorganization is triggered by the mild reduction in cytoplasm pH, a consequent effect of the external low pH, as cytoskeleton remodeling and depolymerization of F-actin under cytoplasm acidification has been reported before (24). Since the drop in the cytoplasmic pH occurs in less then a minute, the gradual change observed in the cytoskeleton, following extracellular acidification, should be related to a slower mechanism, regulated by cytoplasmic pH. A possible candidate is gelsolin, one of the major classes of actin severing proteins. Gelsolin was originally discovered as a factor inducing the gel-sol transformation of actin filaments (25). Gelsolin severs actin filaments and caps the plus ends of actin polymers (26-28), and though normally regulated by calcium ion concentration, it can adapts an active conformation at pH<6.0 (29). In order to further study the involvement of cytoskeletal component involved in PIU, the experiment employed actin cytoskeleton modifiers consisting of calyculin-A, wortmannin, Cytochalasin-B and latrunculin-A. From the data concerning the effects of the different actin cytoskeleton modifiers on PIU, it emerges that there are at least two actin cytoskeletal components that possibly affect PIU. The first are the thick actin cables (stress fibers) that transverse the longitude of the cell between adhesion points and stabilize the cell shape. The second is the cortical cytoskeleton that resides directly beneath the plasma membrane and regulates its shape and deformability. One of the fundamental requirements for the induction of membrane folding appears to be a low level of plasma membrane bending resistance, allowing higher flexibility freedom. It is reasonable to assume that when a cell shape is tightly stabilized by longitude stress fiber or by stronger cortex support, bending or folding the membrane will require a higher deformation force. Same can be said about cells with a high degree of cell-cell interactions, where contact plates increase the structural stability of the culture. According to this view, it can be explained how disassembly of stress fibers enables an increased rate of PIU by allowing the plasma membrane a higher degree of flexibility. The structure and integrity of the cortical cytoskeleton emerges as possibly having a key role in regulating PIU. When severe branching of the cortical actin filaments is induced by cytochalasin-B, the rate of PIU is doubled relative to unperturbed cells. The gradual structural changes seen in cells that have been exposed to low pH, posses similar architecture as those imposed by Cytochalasin-B, i.e. disassembly of stress fibers and severing of cortical actin. A different situation is found when the cells are treated with the phosphatase inhibitors wortmannin and calyculin-A. These modifiers lead on one hand to the disassembly of actin stress fibers which increase membrane bending flexibility but on the other hand cause condensation of the cytoskeletal cortex which decreases membrane flexibility. Indeed, this contradictory effect probably results in a relatively minute impact on PIU increase. Cells treated with latrunculin-A almost completely lost the orderly formation of their cytoskeleton, including the cortical structure. Such disassembly of most of the actin cytoskeleton should remove most conformational restrictions normally imposed on the plasma membrane flexibility and hence be expected to enable a higher PIU rate. The fact that only small PIU enhancement was practically observed, suggests that the cytoskeletal cortex also have a positive role in supporting the progression of PIU, not merely imposing restriction on membrane folding.

The cell's cross-membrane potential difference was found to have a significant affect on the rate of PIU (FIG. 13). The data reveals that de-polarization of the cross-membrane potential is accompanied by two folds increase in PIU relative to cells with unmodified resting potential. The opposite effect of hyper-polarization of the cell's resting potential resulted in a 25% decrease in PIU. A greater hyper polarization is expected to result in a greater reduction in PIU. Microscopic analyses of the cells, under conditions that alter their resting potential, reveal reorganization of the cytoskeleton. Both depolarization and hyper-polarization led to disruption of the longitude actin stress fibers, however while de-polarization induces the formation of thin actin filaments perpendicular to the plasma membrane, hyper-polarization promotes the formation of actin filaments tangent to the plasma membrane. Indeed, previous reports have demonstrated that depolarization of the plasma membrane potential of diverse epithelial cultures, determine reorganization of the cytoskeleton, consisting mainly of disruption of actin stress fiber accompanied by reallocation of peripheral actin toward the cell center (30, 31). Conversely, the cell membrane hyper-polarization was reported to provoke stress fiber disruption and compaction of actin filaments toward the plasma membrane accompanied by an increase in the stability of the adherens junctions (32). It appears that stress fiber disruption and actin condensation adjacent to the plasma membrane have opposite effects on PIU, where the outcome depends on the balance between them. Optionally, materials are provided to cause only one of the fiber disruption and actin condensation, so they cancel each other out to a lesser amount, if at all.

Phospholipids are dipolar in character, because the ester linkages between fatty acids and the glycerol backbones of the membrane lipids are dipolar in character, alignment of these dipoles creates a charge separation which gives rise to the intra-membrane dipole potential, ψ_{d} (33). FIG. 14 demonstrates that in cells that have been treated with chemical agents known to reduce the membrane dipole potential, e.g., Phloretin and CCCP (34, 35), the extent of PIU was higher as compared to normal cells subjected to PIU. Optionally, such materials are used in a physiologically acceptable amount on cells being treated.

Discrete clusters of nanoparticles are seen in the microscopic fluorescence optical cross sections of cells (FIG. 10). Carboxyl modified polystyrene nanoparticles (Φ=60 nm) are seen clustered in discrete sites on the inner side of the plasma membrane or accumulate some way further into the cytoplasm. Unlike dextran polymers, nanoparticles are less susceptible to free diffusion though the cytoplasmic labyrinth-like milieu due to their larger size and therefore are not seen as dispersed as dextran-FITC. Carboxyl coated nanoparticles are better adsorbed to the cell surface than uncoated polystyrene ones even under physiological pH. The adsorption property may be rate limiting for PIU mediated uptake, particularly when it is considered that the particle proximity to the cell surface affects its probability to be internalized. Additional support for this conclusion is found in the data presented in FIG. 6. Small, poly-charged molecules (lucifer yellow) which are less susceptible to adsorb the cell surface then single charged molecules (fluorescein) or polymers (dextran), undergo a lesser extent of PIU.

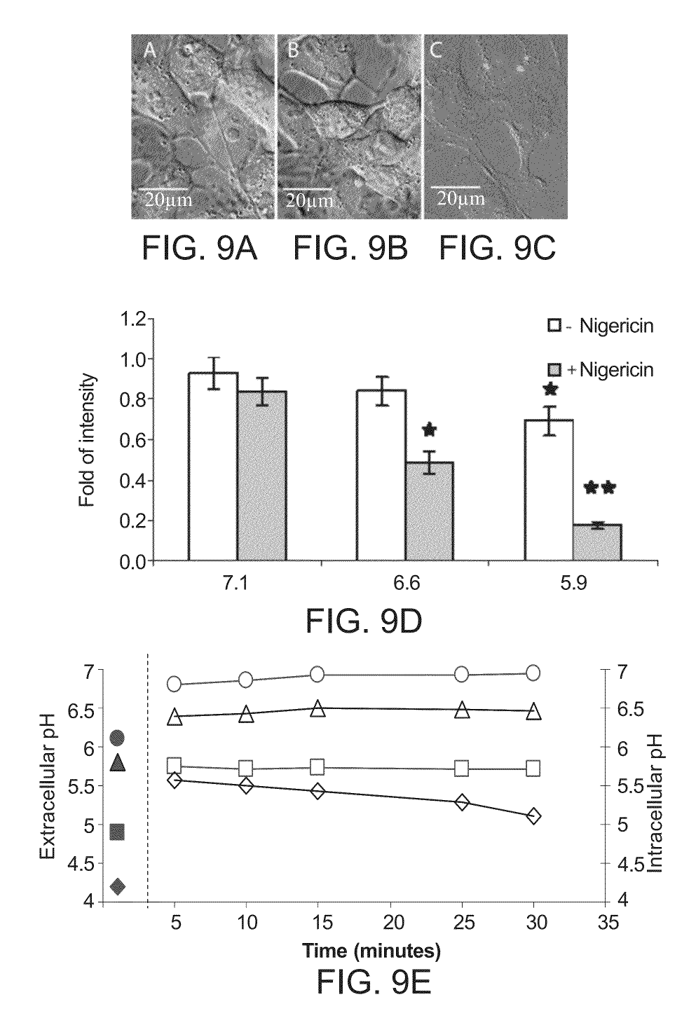

The change in intracellular pH values (cytosol acidity) was evaluated from fluorescence ratio of BCECF-AM, a pH sensitive dye pre-loaded into the cells. The results presented in FIG. 9 demonstrate that cytosol acidity quickly responds both to the decrease in external acidity and to its restoration to a physiological value. It is also apparent from FIG. 9, that when external acidity rises to pH 3.5, the cell ability to maintain constant pH level is (at least) temporarily reduced.

For testing cell viability as function of pH, cells were exposed to low pH for up to 2 hours, thereafter their metabolic activity was analyzed by the alamar-blue assay combined with PI staining for membrane integrity. At near-physiological pH, a fraction of the cell population perishes during the first 30 minutes, whereas the rest of the cells show resilience during the next 90 minutes. At the low pH value of 3.5, the cells keep dying at a linear pace. It is noted that, in general, in some embodiments, the intracellular pH is minimally affected by the extra-cellular pH.

Attenuations in pH sensitive FITC fluorescent intensity were monitored under decreasing extracellular pH. The FITC intensity was found to be independent of the external pH as was verified by flow cytometry and fluorescent microscopy, demonstrated in FIG. 9.

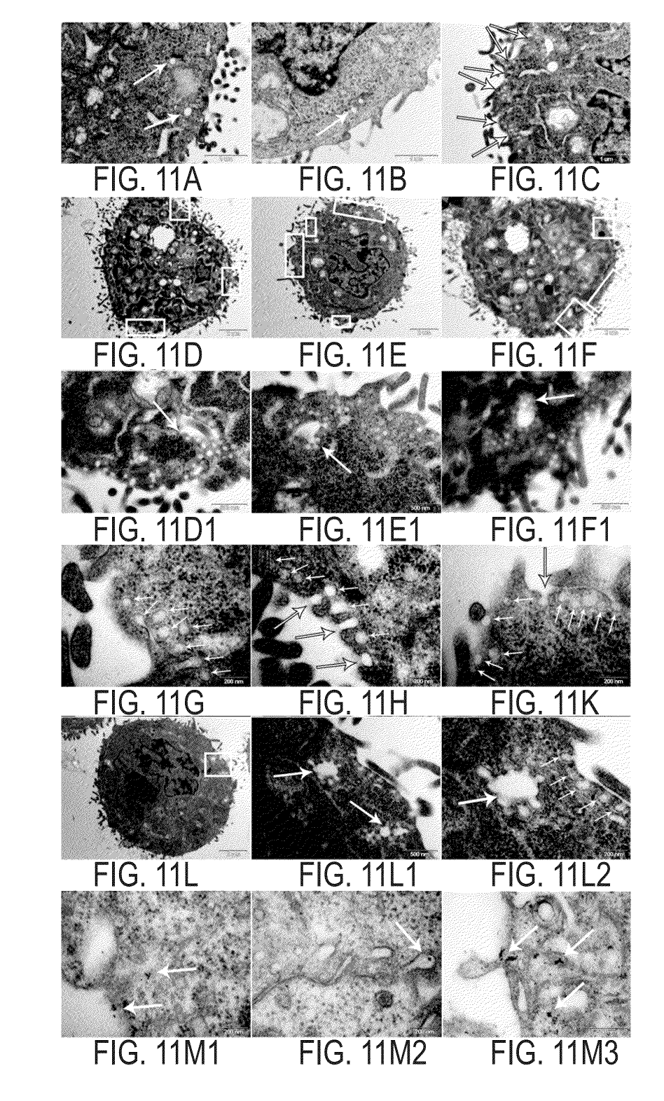

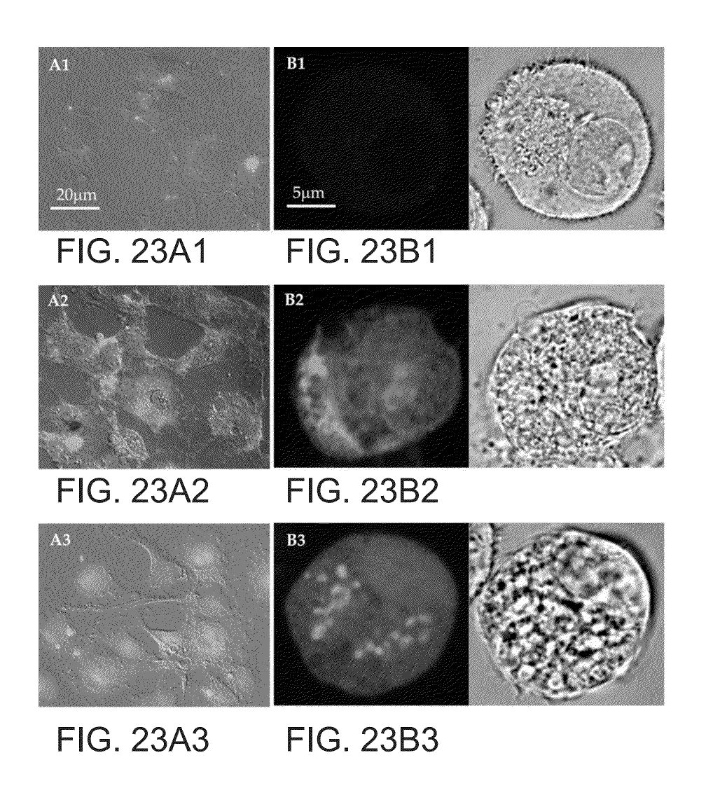

Fluorescence microscopy observations detailed in FIG. 23 show that while the initial appearance of dextran-FITC following PIU is in the form of coarse scattered aggregates, 15 minutes post exposure dextran-FITC can be found homogeneously dispersed throughout the cytosol. These findings are supported by flow cytometry analysis, revealing that 20 minutes following exposure to low pH, intracellular dextran-FITC is not confined to acidic compartments (confirmed by nigericin assay). The intracellular distribution of dextran in the cytosol is better visualized by SCLM where higher optical magnification and optical sectioning of suspended cells (FIG. 23) reveal that shortly after the end of exposure, dextran-FITC can be found both dispersed in the cytosol and concentrated in large structures. The observations portrayed in FIG. 23 seem to be best explained by the formation of acid induced pinocytic vesicles which either release their content into the cytosol, or undergo fusion with each other, forming larger vesicular structures which are not very acidic, based on their FITC intensity. This suggestion is supported by TEM analysis in FIG. 11 which reveals the existence of vesicular structures and invaginations in the vicinity of the membrane, immediately following exposure to the low pH.

### Examples II

### Relating to FIGS. 15-23

These experiments relate generally to examining the relative role of electric field and electric current to the uptake process. For segregating the contribution of the electric field from that of the electric current, cell suspensions exposed to a train of electric pulses were collected from the proximity of either a cathode, an anode or from a middle section of a three chamber membrane separated device (see FIG. 15), analyzed and compared. The findings suggest that the uptake process is driven by electrochemically induced acidification at the anode-medium interface.

Material and Methods

Chemicals:

Sodium ascorbic acid (SAA), Bis-Dehydroascorbic acid (DHA), BSA-FITC, dextran-FITC, dextranase, propidium iodide (PI), hydrochloric acid (HCl), lucifer yellow (LY) tetramethyl-benzidine (TMB), and hank's balanced salt solution (HBSS) were purchased from Sigma. Trypan-blue (TB), phosphate buffered saline (PBS) and 4-(2-Hydroxyethyl)piperazine-1-ethanesulfonic acid (HEPES) were purchased from Biological industries. Dichlorodihydrofluorescein diacetate (H₂DCF-DA) was purchased from invitrogen.

Cell Culture:

COS 5-7 cells (fibroblast-like cells, African green monkey kidney derived from CV-1 subclone of COS 7) were cultured in Dulbecco's Modified Eagle Medium (DMEM), supplemented with L-glutamine (2 mM), 10% FCS and 0.05% PSN solution. All cells were grown in 75 cm² tissue culture flasks (Corning) at 37° C., in a humid atmosphere of 5% CO₂ in air. Cells were harvested before reaching ˜80% confluence by using 0.25% trypsin solution (with 0.05% EDTA) for 5 min at 37° C. The cells were centrifuged (1 min at 400g, by Sorvall RT6000D), their solution aspirated and were re-suspended in growth medium. All culture media, antibiotics, trypsin and serum products were purchased from Biological Industries (Beit Haemek, Israel).

Exposure Set-Up to Low Electric Fields

Exposure of cells to low-intensity trains of unipolar rectangular voltage pulses was carried in a three-compartment exposure set-up where the anode and cathode were connected to an electric pulse generator (Grass S44 Stimulator). The exposure set-up consisted of a rectangular chamber made from polystyrene, 15 mm long, 10 mm wide, with 0.5 cm² area platinum electrodes positioned on the extreme sides (see FIG. 15). The chamber is divided by two porous membranes (PolyEtherSulfon, 0.8 μm pores, 200 μm depth), into three distinct and equal compartments: anode, cathode and center ones. The electric field parameters applied were monitored on line by recording the voltage and the current (by means of a wide band current probe, Pearson) on an oscilloscope. Typically, a cell suspension (1*10⁶ cells/ml) is exposed to a train of electric pulses consisting of unipolar rectangular pulses with duration of 180 μs, frequency of 500 Hz for the total time of exposure of one minute.

In other embodiments of the invention, other pulse shapes such as a triangular or sine or sawtooth or arbitrary pulse shape can be used and/or longer pulse width and/or applied for longer time period. Optionally, a biphasic pulse or charge balanced sequence is used, which pulse form can undo some polarization effects, while not undoing the uptake. In some embodiments, an AC pulse is used, optionally selected so that uptake has time to occur during an anodic phase. Optionally, the pulse parameters are selected so that enough charge is provided to ensure a desired pH (e.g., within a range), optionally taking into account buffering and/or flow affects.

No significant transport of electrolytic products or charged low molecular weight dyes could be detected from the anode or cathode compartments into the central one during one minute of exposure to EPT. The central compartment was monitored for the presence tryphan blue or lucifer yellow by monitoring absorption and fluorescence (430/530), respectively. The passage of hydrogen ions and oxidative intermediates into the central compartment was monitored by pH electrode and by TMB color change, respectively.

Uptake Studies

Cells (COS 5-7) were harvested, re-suspended in DMEM (1 mg/ml glucose, 5% FCS), and incubated (37°, 5% CO₂) as designated by the experiment. For uploading the cells with either H₂DCF-DA (20 μM) or Bis-DHA (1 mM) incubation lasted 90 minutes. When no pre-treatments were required, the cells were kept in the DMEM for 30 minutes. Following incubation, the cells were centrifuged at 400g for 1 min, the solution aspirated, and cells re-suspended in HBSS exposure media.

Cells in the test groups were exposed to electric pulse train (EPT) in the presence of dextran-FITC (70 kDa, 1:250 FITC:glucose ratio, Sigma-Aldrich) for 60 seconds at room temperature (22-26° C.), immediately diluted with 5 fold larger volume of cold DMEM-H (25 mM HEPES supplemented DMEM without phenol red) and kept on ice.

Exposure of cells to pulse acidification was through the addition of HCl to the HBSS suspension for 60 seconds, terminated by the addition of a three fold larger volume of DMEM-H.

Cells in the control group were incubated with dextran-FITC for 60 seconds at room temperature, after which they were moved to ice as described above.

For determining the fraction of the fluorescent probe which adsorbed to the cells membrane, cell suspension were pre-cooled to 4° and exposed to EPT in this temperature to eliminate endocytosis. For measuring the cell auto-fluorescence, EPT exposure was carried in the absence of an external fluorescent probe.

Analysis:

Cell suspensions were centrifuged (400g for one minute), their solutions aspirated and they were re-suspended with 0.5 ml DMEM-H supplemented with 10u/ml dextranase (Sigma-Aldrich) for 10 minutes at RT. Cells were centrifuged again; their solution aspirated and they were re-suspended with 0.2 ml DMEM-H. PI (25 μg/ml) as a label for permeabilized cells and tryphan blue (0.01% w/v) as a fluorescence quenching agent for extracellular FITC (36) were added to each sample shortly before analysis

Flow cytometry analysis was carried out by FACSort (Becton @ Dickson, San Jose, Calif.), employing a 488-nm argon laser excitation. The green fluorescence of FITC was measured via 530/30 nm filter, the red fluorescence of PI was detected via a 585/42 nm filter. To eliminate signals due to cellular fragments, only those events with forward scatter and side scatter comparable to whole cells were analyzed. Ten thousand cells were examined for each sample and data were collected in the list mode. The analysis of flow cytometry data was performed using WINMDI 2.9 software (Joe Trotter, The Scripps Research Institute). For each sample the geometrical mean was calculated without including PI stained cells. Cell intensity was determined as fold of induction relative to cells' constitutive uptake.

Fluorescent microscopy is used to verify that no visible dextran-FITC is left adsorbed to the cell's membrane.

Determination of Intracellular ROS

Harvested cells were incubated with H₂DCF-DA (20 μM for 90 minutes 5% CO₂ at 37°). Following the loading process the cells were washed once with HBSS before exposure to EPT. H₂DCF-DA is a non-fluorescent form of fluorescein, passively permeating into the cell where it is cleaved by cytosolic esterase to H₂DCF. It gains its fluorescent properties when oxidized into DCF. DCF intensity was analyzed by FACS using the 530/30 nm channel.

Determination of Electrolytic Products

In order to determine the EPT induced oxidative stress in the extracellular media, TMB was employed, a colorless solution that transforms into blue when oxidized. TMB was added to the media before exposure to EPT. The solution was collected and was stabilized with 1.25M HCl, further transforming its color to yellow. Light absorbance was measured using Tecan GENios plate reader at 450 nm.

Evaluation of pH value in the medium shortly after exposure was determined by pH electrode. For qualitative evaluation of the transient pH formed at the anode surface during EPT, pH paper indicators prepared by cutting 5 mm by 7 mm rectangular section from pH strips (PANPEHA, Sigma-aldrich) were used according to their designated pH range. The pH indicator paper is placed perpendicular to the electrode plane and in one quick movement dipped in and pulled out from the anodic compartment solution, then gently placed on an adsorbent paper facing down. Osmolarity was measured using an osmometer.

Medium Conductivity

Lowering medium conductivity was achieved by replacing some of the soluble ions with sucrose. 300 mM sucrose solution in water was used to dilute HBSS solution at several ratios to final sucrose concentrations from 200 mM down to 50 mM.

Annexin

Harvested cells were exposed to ELpH for one minute. Cells in the control group were exposed to the apoptotic agent staurosporine (Sigma). Cells were centrifuged (1 min at 400g) and suspended in an annexin-FITC buffer solution (Sigma). Annexin-FITC were added along with PI for a 15 minutes incubation before FACS analysis.

Statistics

Statistical analysis was performed with student's t-test and ANOVA, using Microsoft Excel.

Results

Dependence of Electric Induced Uptake on Anodic Current Density

In order to differentiate between cells exposed mostly to the electric field and those exposed in addition to the electrolytic products formed during the exposure to EPT a three-compartment device consisting of a central compartment, an anode compartment and a cathode compartment was constructed. Following exposure to a train of electric pulses (20V/cm, 200 mA/cm²), a 10 fold increase in cellular uptake of dextran-FITC is detected in the anodic compartment only, while changes in uptake of the cells in the other compartments are not significantly different from the constitutive one (FIG. 16).

The relative contributions of electrical field and electrical current to the elevated uptake of dextran-FITC was examined by modifying electric conductivity of the medium through substituting ions in the media with sucrose. The results presented in FIG. 17 show that the electric current has a far greater impact on the extent of dextran-FITC uptake as compared to that of the electric field.

Dependence of Electric Induced Uptake on ROS Formation

Electric current promotes hydrolysis and oxidation at the anodic side, consequently inducing the production of radical oxidative intermediates. The term oxidative stress (OS) refers to an imbalance between production of reactive species (ROS) and their disintegration, leading to elevated oxidative activity and damage (37). Such oxidative stress, either extra or intra-cellular, could play a part of the electric induced uptake of macromolecules. In order to moot this possibility, OS was examined in the three-compartment device, monitored by the color conversion of TMB. Oxidation occurs during the exposure to EPT (20V/cm, 200 mA/cm²) only near the anode surface. Addition of TMB immediately after the termination of the EPT did not lead to its color conversion. Addition of 2 mM sodium ascorbic acid (SAA) to the medium containing the TMB prior to electric exposure prevented its oxidation during the exposure.

The intracellular OS level was monitored by H₂DCF, a non-fluorescent probe that is oxidized by radical hydroxyls into the fluorescent DCF form. Following the cells pre-loading with H₂DCF, OS levels, as monitored by intracellular DCF fluorescence intensity, are elevated only in those cells suspended and exposed to EPT in the anode compartment (20V/cm, 200 mA/cm²). Upon addition of SAA (2 mM) to the external medium, the intracellular OS declines by 66% (FIG. 18). Alternatively, pre-loading the cells with DHA, a reduced form of ascorbic acid whose entry into the cell is facilitated by GLUT receptors (38), was sufficient to abolish the EPT induced increase of the DCF fluorescent intensity (FIG. 18).

The presence of either intracellular or extracellular ascorbic acid during exposure to EPT, at concentrations that were shown to suppress intracellular OS, had a minor insignificant effect on the extent of EPT induced dextran uptake (FIG. 19).

Dependence of Electric Induced Uptake on the Anodic Acidification of the Extracellular Compartment

Hydrolysis is responsible for lowering pH values (acidification) near the anode and elevating it (alkalization) near the cathode. Osmolarity and pH values of HBSS medium (with 100 mM HEPES) taken from the anode compartment was measured soon after it has been exposed to EPT (20V/cm, 200 mA/cm²) and was found unchanged (pH 7.5, 290 miliOsmol). However, using a pH sensitive indicator in the anode compartment during the actual application of the electric current, demonstrates the existence of a transient, pH 1.5 zone near the electrode face, whose width is inversely dependent on buffer concentration (FIG. 20). In some embodiments, such positioning (between cells and anode) is controlled to achieve a desired LpHU effect. Optionally or alternatively, a pH sensitive indicator is used to calibrate the operation of a reactor, for example, as described above (e.g., system 2700).

Decreasing the concentration of HEPES buffer in the exposure medium, before applying a constant current density (200 mA/cm²), strongly increases the level of dextran uptake, with the major effect taking place between 80 mM to 70 mM HEPES concentration (FIG. 21). This elevation of cell uptake could be the result of the widening of low pH zone, engulfing a greater portion of the cell population in the anodic compartment.

For simulating the effect that EPT induced extreme low pH (ELpH) exerts on intracellular uptake of fluid-phase dextran, cells were suspended in HBSS without additional buffers, and subjected to ELpH by adding HCl to a cell suspension containing either dextran-FITC or lucifer yellow. After one minute incubation, cell suspensions were immediately diluted with 3 fold larger volume of cold buffered DMEM to restore pH to a normal pH of 7.5.

As noted above with reference to FIGS. 1A-1C, the relation between pH level and cellular uptake of fluid phase dextran-FITC is typically characterized by a gradual response from pH 7 to pH 4, and a very steep elevation of uptake starting at pH 4 and reaching saturation at pH 3.

The possible effect of osmolarity change imposed by the addition of HCl to the suspensions was evaluated by replacing HCl with the same concentration (50 mM) of NaCl. Under these conditions no change in uptake was observed.

To ascertain that the increase in cell fluorescence is indeed intracellular, cells exposed to ELpH in HBSS solution with dextran-FITC, were visualized and optically sectioned by confocal fluorescent microscope (Zeis axiovert). The 488 nm/530 nm fluorescence was acquired and found to associate with the cytosol (FIG. 23).

Since low pH values are potentially destructive for cell integrity, necrosis is evaluated by analyzing PI permeability as a measure of membrane integrity. 10% of the cell populations analyzed by FACS were found positively PI stained from 10 minutes to 2 hours after ELpH exposure. Cells that were exposed to ELpH were seeded in culture flasks and incubated for 24 hours at 37° with 5% CO2 atmosphere, grow to the same extent as unexposed control cultures. The initiation of apoptosis is determined by FACS analysis of annexin-FITC binding to the outer membrane leaflet of the cells. Annexin binding to the plasma membrane was not found to increase during the 2 hours following the exposure to ELpH, unlike those cells exposed to staurosporine.

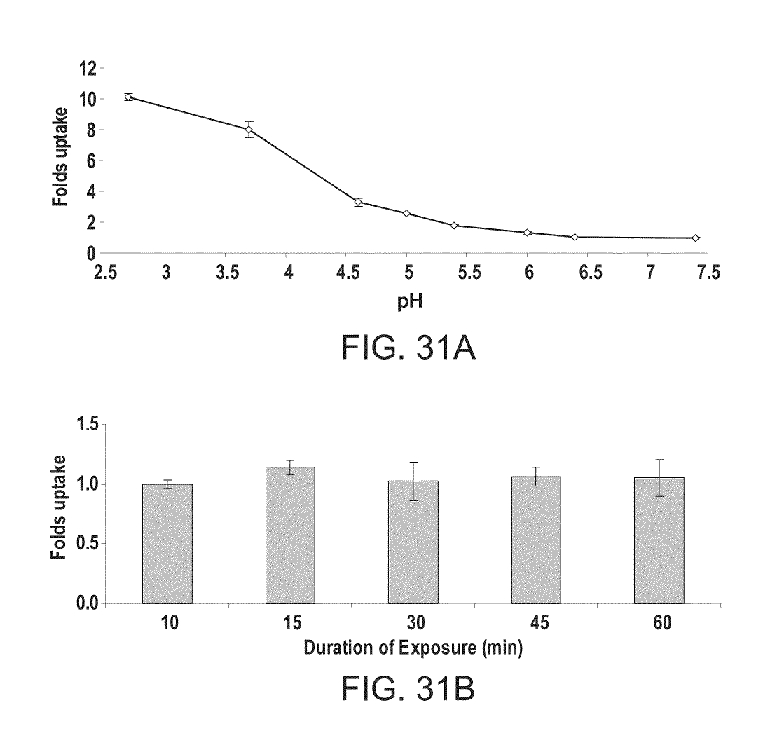

### FIGS. 31 to 37

Cell based drug delivery systems are assumed, in some cases, to possess a number of advantages including prolonged delivery times and biocompatibility. These systems could be especially efficient in releasing drugs in blood circulations for weeks, can be easily processed and could accommodate traditional and biologic drugs. Advances in this field have been restricted by the inefficiency of existing methods for loading erythrocytes and by the lack of methods to load nucleated cells. Thus, to date very little clinical advance in managing complex pathologies has been made, especially when side effects become serious issues.

The methods of encapsulation seek an enhanced performance of the substance encapsulated, whilst ensuring that the erythrocytes undergo the fewest possible alterations, so that in functional terms it will as similar as possible to a normal erythrocyte. This requirement is vital for ensuring the proper survival and circulation of the loaded erythrocytes.

We induce uptake in RBC based on the methods we discovered when exposing cells to high proton concentration. The dependence of dextran uptake on external pH appears to take a sigmoid-like shape (FIG. 31A). Significant levels of uptake relative to RBC autofluorescence at physiological pH 7.4, can be seen only from pH<6 (P<0.05, one tail t-test). Pulse labeling studies of the uptake rate suggest that the same rate is maintained at a constant level (P>0.05, ANOVA), irrelative to the length of preceding acidification (FIG. 31B).

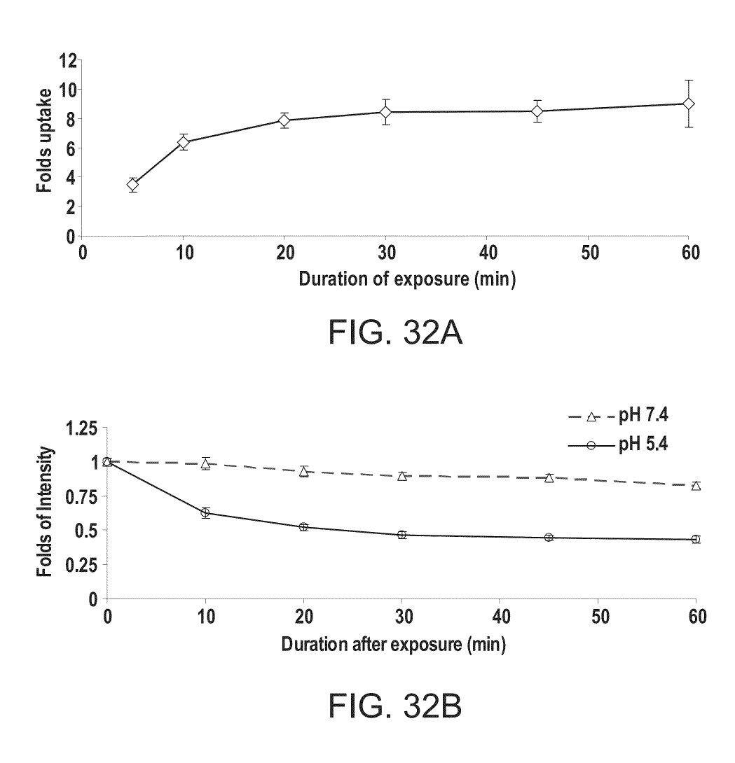

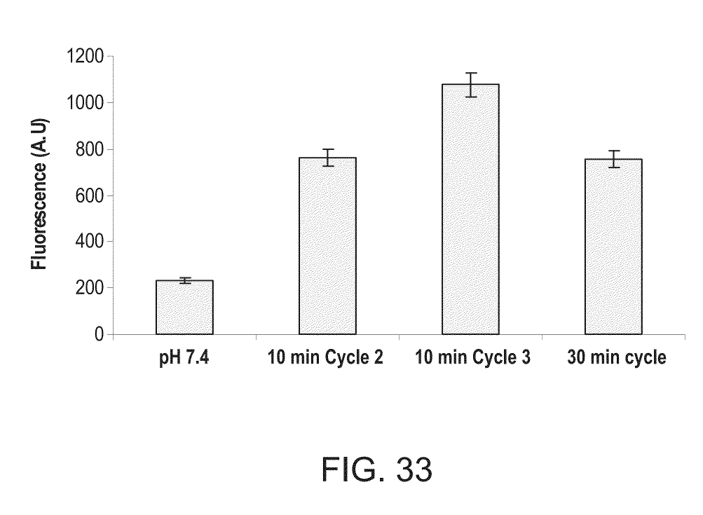

The uptake kinetics of dextran-FITC by in RBCs at pH 5.4 (FIG. 32A) demonstrates a saturating curve possessing an IC₅₀ between 5 and 10 minutes of exposure. This apparent saturation may be attributed to a competitive efflux process. To examine this possibility, RBCs were first exposed for 10 minutes to pH 5.4 in the presence of dextran-FITC. Next, the cells were washed and re-suspended in fresh PBS-G of either pH 7.4 or 5.4, in the absence of dextran and then incubated at 37° C. for durations of 10, 20, 30, 45 and 60 minutes, followed by flow cytometry analysis (FIG. 32B). Dextran concentrations in RBCs incubated under physiological conditions (pH 7.4) suffers some gradual 20% decrease in value for 60 minutes after PIPC(P<0.05, one tail t-test). However, when RBCs are incubated at pH 5.4, the dextran concentration decline rapidly during the first 10 minutes, losing 50% of their initial value after 20 minutes and continue to decrease at a declining rate for the rest of the period monitored. It appears that long exposure of RBCs to low pH inflict some damage to the cells ability to retain the uptake cargo molecule (FIG. 32b). In an exemplary embodiment of the invention, RBCs are instead subjected to a pulsed treatment regime. FIG. 33 demonstrates that exposing the cells to three cycles of 10 minutes exposure, produce a higher concentration of intracellular dextran-FITC then one exposure of 30 minutes. In an exemplary embodiment of the invention, pulsed uptake is used to avoid/reduce causing leakage in cells and thus reduce efflux. Optionally or alternatively, cells that are sensitive to low pH can be treated. For example, one hour exposures separated by two hour rest periods may be used for siRNA transfection of pH-sensitive cells. The actual length of rest periods and uptake periods may depended, for example, on the rate of cell death (as function of pH), uptake (as function of pH), desired yield and/or time allowed for the total treatment.

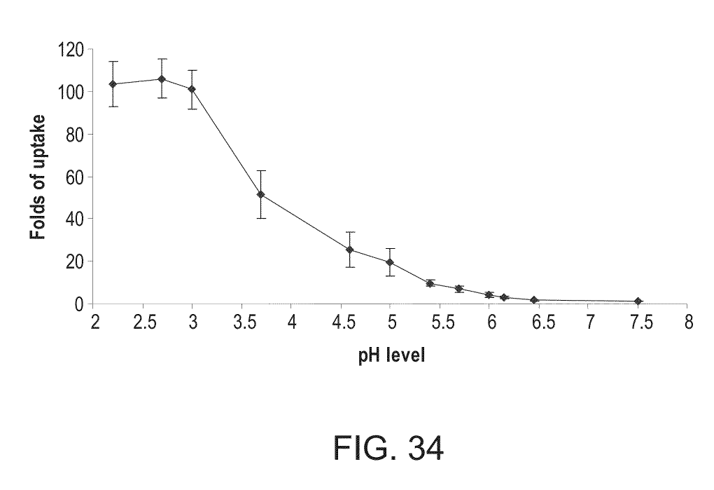

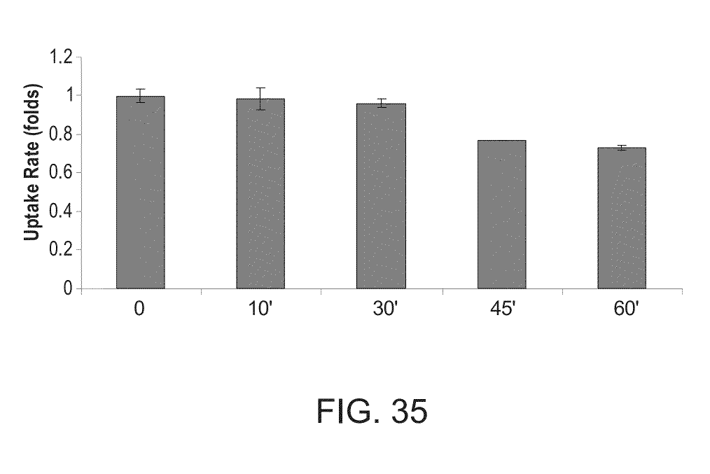

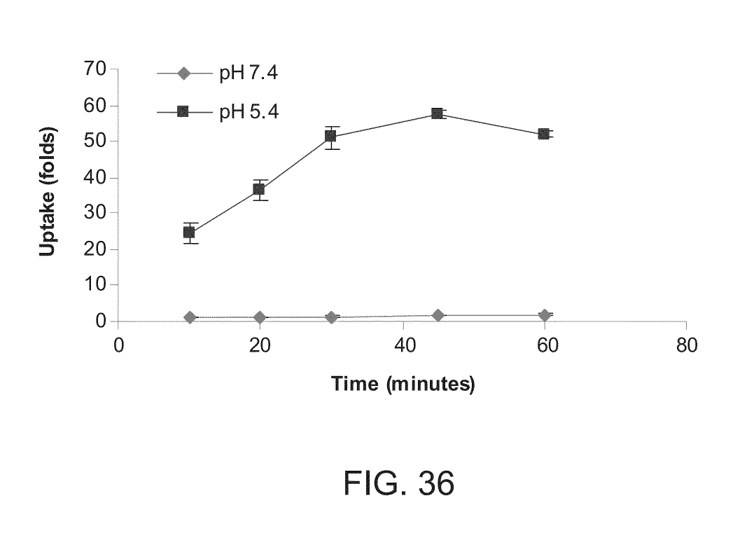

Lymphoblast cells (TK6 line) were grown in suspension and exposed to low pH solution in the presence of dextran-FITC. PIU of dextran-FITC as a function of the external pH, portrays a sigmoid-like relationship, with the steepest rise observed in the range of 3<pHₑₓₜ<4, for three different cell lines (FIG. 34). The relative high extent of PIU in TK6 cells could be attributed to the higher exposure of their cell surface to the macromolecules, due to them being suspended cells. The kinetics of PIU mediated uptake of dextrans by TK6 cells emerges as having a constant rate, reflected in the pulse labeling studies (FIG. 35). However, the kinetics of dextran accumulation in the cells could take the form of saturation curve under increasing exposure durations (FIG. 36).

General

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of inducing uptake in living cells, comprising:
determining a desired uptake of a non-protein material by the cells; and
in response to said determining, temporarily subjecting said cells to a local chemical environment which encourages inward vesiculation or invagination of a plasma membrane of the living cells causing said uptake and which chemical environment does not substantially affect said cells by osmotic effects, said subjecting being for a period of less than 6 hours.

2. A method according to claim 1, wherein said encouraging local chemical environment comprises a reduction in pH which is not local to the membrane of the cells.

3. A method according to claim 1, wherein said encouraged inward vesiculation comprises vesiculation caused by a chemical effect and not by a biochemical effect involving the chemical activity of proteins.

4. A method according to claim 1, wherein subjecting comprises intentionally subjecting to an environment which causes substantial cell death when subjecting for a period greater than said period.

5. A method according to claim 1, comprising selecting method parameters in accordance with a pH mediated uptake mechanism.

6. A method according to claim 1, wherein said subjecting comprises controlling an uptake rate of said cells by controlling said local chemical environment.

7. A method according to claim 1, wherein said subjecting comprises avoiding damaging the living cells by said uptake and by materials being taken up.

8. A method according to claim 1, wherein said subjecting comprises avoiding damaging the living cells by said uptake.

9. A method according to claim 1, wherein said subjecting comprises applying an anodic current for a time and amount sufficient to cause acidification by anodic hydrolysis of water, to said cells to provide or modify said encouraging chemical environment, said current not being sufficient to cause substantial electroporation.

10. A method according to claim 9, wherein applying comprises applying a voltage and current density sufficient for electrolysis for a duration of between 1 second and 15 minutes.

11. A method according to claim 9, wherein applying comprises controlling an applied pH to avoid applying a pH to cells below a desired level, by positioning of an anode.

12. A method according to claim 1, where said local chemical environment comprises an increase in hydrogen ions, wherein said environment has a pH value between 3 and 6 and wherein said increase is to above physiological concentrations of hydrogen ions, for a time period which does not kill more than 25% of said cells.

13.-15. (canceled)

16. A method according to claim 12, where said increase is provided by one or more of:
a. adding a soluble formulation of acidic material;
b. release of hydrogen ions from solid, semi-solid or liquid substances;
c. release of hydrogen ions from a proton exchange membrane (PEM); and
d. chemical cleavage of molecules to release hydrogen ions.

17. A method according to claim 1, where said living cells are inside a living body.

18. (canceled)

19. A method according to claim 1, wherein said local environment is effected by the provision of one or more formulations to said environment.

20.-21. (canceled)

22. A method according to claim 1, comprising providing at least one agent to be introduced into said cells by said uptake.

23. A method according to claim 22, wherein said agent comprises a simple molecule formulation.

24. A method according to claim 22, wherein said agent comprises a nanoparticle.

25. A method according to claim 22, wherein said agent is selected from a group consisting of:
g. a nucleic acid agent;
h. a small molecule agent;
i. a protein aceous agent; and
j. a carbohydrate agent;
k. lipid agent; and
l. a combination of same.

26.-32. (canceled)

33. A method according to claim 1, comprising inducing said uptake for one or more of the following purposes:
a. interacting with cell functions including one or more of enzymes, catalytic domains and respiration chain components;
b. incorporate toxins, peptides, proteins, fatty acids, inhibitors, blockers or promoters;
c. introducing to said cells new properties, new functions, correcting resident mutations or silencing existing functions;
d. interfering with protein expression and cell functioning, for example anti-sense RNA and siRNA;
e. labeling structures in the cell;
f. identifying biological pathways; and
g. inducing cell proliferation, growth arrest or cell killing.

34. A method according to claim 22, comprising, selecting said formulation to have a desired therapeutic effect.

35. (canceled)

36. A method according to claim 22, comprising maintaining a desired level of said formulation in said living cells, by said uptake.

37. (canceled)

38. A method according to claim 22, comprising maintaining a desired level of said material in said living cells, by interfering with expulsion of said material from said cells.

39. A method according to claim 1, wherein said cells are red blood cells.

40. A method according to claim 1, wherein said cells are white blood cells.

41.-42. (canceled)

43. A method according to claim 1, wherein said duration is less than 30 minutes.

44. (canceled)

45. A method according to claim 1, comprising modifying a mechanical stiffness of said cells for said uptake.

46. (canceled)

47. A method according to claim 1, wherein determining comprises calculating and imposing a chemical environment designed to provide said uptake to within a factor of 4 of said desired uptake.

48.-51. (canceled)

52. A method according to claim 1, wherein said uptake is at least 10 times an uptake in a neural chemical environment.

53.-56. (canceled)

57. Apparatus for controlling cellular uptake, comprising:
at least one electrode;
a power source, electrifying said at least one electrode as an anode; and
a controller adapted to control said power source according to a pH-mediated uptake protocol for non-protein materials.

58. Apparatus according to claim 57, comprising a pH sensor.

59. Apparatus according to claim 57, comprising a tissue displacer located adjacent said electrode.

60. Apparatus according to claim 57, wherein said controller is configured to estimate an uptake effect based on said protocol.

61. Apparatus according to claim 57, comprising a source of formulation for said uptake.

62. A method of processing blood, comprising:
providing blood;
causing uptake of material into cells of said blood using a pH mediated effect;
stopping said uptake.

63. A method according to claim 62, comprising returning said treated blood to a body from which it was taken.

64.-70. (canceled)
